SEQUENCE LISTING (2 sequences)

SEQ ID NO: 1 - DNA, 30 bp, Artificial Sequence
  Forward Primer
cttgtcgacgccaccatgcacttgcgactg

SEQ ID NO: 2 - DNA, 32 bp, Artificial Sequence
  Reverse Primer
gtcgagaattcttatcacttatcgtcgtcatc

(19) United States
(12) Patent Application Publication
(10) Pub. No.: US 2023/0338466 A1
(43) Pub. Date: Oct. 26, 2023

(54) COMPOSITIONS AND METHODS COMPRISING R-SPONDINS FOR TREATMENT OF TUMORS

(71) Applicant: CHILDREN'S HOSPITAL MEDICAL CENTER, Cincinnati, OH (US)

(72) Inventors: Yuting Tang, Cincinnati, OH (US); Gang Huang, Cincinnati, OH (US)

(21) Appl. No.: 18/007,771

(22) PCT Filed: Jun. 1, 2021
(86) PCT No.: PCT/US21/35141

§ 371 Date: Dec. 2, 2022

Related U.S. Application Data
(60) Provisional application No. 63/034,010, filed on Jun. 3, 2020.

(51) Int. Cl. A61K 38/17; A61P 35/00; A61K 39/395

(52) U.S. Cl. CPC A61K 38/1709; A61P 35/00; A61K 39/3955; A61K 2039/505

(57) ABSTRACT

Disclosed herein are methods for treating a cancer in an individual, including administration of an R-spondin protein, for example one or more of R-spondin1, R-spondin2, R-spondin3, R-spondin4, and combinations thereof. Further disclosed are methods of treating an individual having a cancer, the method including determining an R-spondin level in the individual, and administering a treatment to the individual.

### CROSS-REFERENCE TO RELATED APPLICATIONS

This application claims priority to and benefit of U.S. Provisional Application Ser. No. 63/034,010, filed Jun. 3, 2020, entitled “R-Spondins Enhance NK-cell Immunity,” the contents of which are incorporated in their entirety for all purposes.

### STATEMENT REGARDING FEDERALLY-SPONSORED RESEARCH

This invention was made with government support under DK105014, CA248019, DA038017, AI148080, AR073228, awarded by the National Institutes of Health. The government has certain rights in the invention.

### BACKGROUND

Treatment for cancers, for example solid tumors, include the use of immune checkpoint therapies, which target pathways in T cells and NK cells to promote an anti-tumor immune responses. To this end, clinical care of cancer patients has advanced with approval of anti-CTLA-4 and anti-PD-1 antibodies such as ipilimumab, pembrolizumab, and nivolumab. While anti-PD-1/PD-L1 antibodies have advanced treatment in patients with solid tumors, response rates are relatively low, and treatment of cancers, in particular solid tumors, is in need of improvement.

Thus, there remains a need to develop new cancer immunotherapies that induce adaptive immune response against tumors or target the immunosuppressive immune cells.

### BRIEF SUMMARY

Disclosed herein are methods for treating a cancer in an individual, including administration of an R-spondin protein, for example one or more of R-spondin1, R-spondin2, R-spondin3, R-spondin4, and combinations thereof. Further disclosed are methods of treating an individual having a cancer, the method including determining an R-spondin level in the individual, and administering a treatment to the individual.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A-H. EC- and CAF-derived R-spondins correlate with anti-cancer immune cell signatures and prognosis in multiple cancers. (1A) Hierarchically clustered Kendall's correlation matrices using the indicated datasets from TCGA database based on components of Wnt signaling pathway (Table 1) and NK-cell signature genes (KIR2DL4, NCR1, KLRD1, KLRC1, KLRC2, KLRC3, KLRC4, KLRB1, KLRK1). (1B) Heatmap visualization of the gene expression levels of RSPO1, RSPO2, RSPO3, and RSPO4 in different cancer tissues (T) and the matched normal tissues (N) in the TCGA datasets. Data are shown as log 2 (TPM+1). (1C) Results of Spearman's rank correlation analyses of RSPO3 with NK-cell signature genes using TCGA datasets plotted with coefficient R and −log₁₀ (P-value). Cancer types with correlation P-value<0.01 and R>0.45 are marked red. (1D) Overall comparison of Spearman's rank correlation coefficients for RSPO3 with NK-cell signature for a total of 19 types of tumors and normal tissues. Wilcoxon tests were performed. (1E) Spearman's rank correlation plots for RSPO3 with CD69, GZMA, GZMB, and IFNG in SKCM and PAAD of TCGA datasets. (1F) Kaplan-Meier curves showing the prognostic impact of RSPO3 expression levels for the overall survival of patients with SKCM and classical PAAD of TCGA datasets. Hazard ratios (HR) and P values of Log-rank tests are shown. Patients were grouped by the median expression levels of RSPO3. (1G-1H) The expression of the detectable RSPO genes in the single-cell RNA-seq datasets of melanoma (1G) and pancreatic carcinoma (1H) patients reported previously. (H) Representative pictures of human melanoma tissues with immunohistochemical staining of RSPO3 and CD31 in serial sections. Pictures are shown as 200×. Abbreviations for the cancer types are listed in Table 1.

FIG. 2A-2H. LGR6 is prominently expressed by human NK cells. (2A) The expression of LGR4, LGR5, LGR6 in the single-cell RNA-seq dataset of human melanoma reported previously accessed from Broad Institute's Single Cell Portal. (2B) Visualization of LGR4, LGR5, LGR6, and NCAM1 expression in human immune cell subtypes in DICE datasets. (2C) Heatmap visualization of the gene expression levels of LGR4, LGR5, and LGR6 in bulk RNA-seq dataset of human circulating NK cell subsets reported previously. (2D) Quantitative RT-PCR analysis of LGR4 expression for different immune cell types isolated from human peripheral blood pooled by two different donors. (2E) Protein expression of LGR4 in different immune cell types isolated from human peripheral blood from two different donors. (2F) Quantitative RT-PCR analysis of LGR6 expression for different immune cell types isolated from human peripheral blood pooled by two different donors. (2G) Protein expression of LGR6 in different immune cell types isolated from human peripheral blood from two different donors. (2H) Spearman's rank correlation plot for RSPO3 with LGR6 using TCGA SKCM dataset. Data are shown as mean±s.d. for 2D and 2F.

FIG. 3A-3Q. Exogenous expression of R-spondin3 in the TME inhibits tumor progression. (3A-3B) Growth curves of B16F10-EV and B16F10-Rspo3 in NRG mice (n=8 mice per group) (3A) and representative pictures of tumors dissected on 18 days after inoculation (3B). (3C-3D) Growth curves of B16F10-EV and B16F10-Rspo3 in syngeneic B6 mice (n=8 mice per group) (3C) and representative pictures of tumors dissected on 18 days after inoculation (3D). (3E) Survival curves and result of the Log-rank test for growth of B16F10-EV and B16F10-Rspo3 in syngeneic B6 mice (n=11 for each group). (3F-3G) Growth curves of Pan02-EV and Pan02-Rspo3 in NRG mice (n=8 mice per group) (3F) and representative pictures of tumors dissected on 33 days after inoculation (3G). (3H-3I) Growth curves of Pan02-EV and Pan02-Rspo3 in syngeneic B6 mice (n=8 mice per group) (3H) and representative pictures of tumors dissected on 33 days after inoculation (3I). (3J) Survival curves and result of the Log-rank test for growth of Pan02-EV and Pan02-Rspo3 in syngeneic B6 mice (n=12 for each group). (3K) Experimental design for panels 3L-3N. Recombinant mouse R-spondin3 (10 μg) or the same volume of PBS were intratumorally injected to B16F10 tumors in syngeneic B6 mice (n=6 mice per group). (3L) Tumor volumes measured before and 2 days after the 1^{st} dose of R-spondin3 i.t. therapy were compared. Two-way ANOVA test with Sidak's multiple comparisons. (3M) Tumor growth curves. (3N) Survival curves and result of the Log-rank test. (3O) Growth curves of Pan02-EV and Pan02-Rspo3 cells in Lgr6^{-/-} mice (n=5-7 mice per group). (3P-3Q) Tumors of Pan02-EV or Pan02-Rspo3 cells grown in wild-type or Lgr6−/− mice were dissected 35 days after inoculation. Tumor weights (3P) and representative pictures of tumors (3Q) were shown. For 3A, 3C, 3F, 3H, 3M, 3O, and 3P, two-way ANOVA test with or without Tukey's multiple comparisons tests were performed. Data are shown as mean±s.e.m or individual sample results. Data are representative of at least two independent experiments. P<0.05 is considered as statistically significant. ns, not statistically significant, *P<0.05, **P<0.01, ***P<0.001.

FIG. 4A-4L. Exogenous expression of R-spondin3 in the TME enhances anti-tumor immunity. (4A) Absolute numbers of tumor-infiltrating NK cells (CD45⁺CD3⁻NK1.1⁺DX5⁺) in B16F10-EV and B16F10-Rspo3 tumors (n=5-6 mice per group) by flow cytometry analysis. (4B) Representative flow plots (left) and summary (right) of the percentages of tumor-infiltrating NK cells expressing Granzyme B, perforin, and CD69 in the B16F10-EV and B16F10-Rpso3 tumors (n=5-7 mice per group). (4C) Cytotoxicity of freshly isolated tumor-infiltrating NK cells from B16F10-EV and B16F10-Rspo3 tumors was measured with chromium (51Cr)-release assays using YAC-1 cells as target cells with the indicated E: T (effector: target ratios). Purified NK cells were pooled from four mice per group. (4D) Percentages of CD103+DC (lineage [CD90.2, CD45R, Ly6G, NK1.1]⁻, CD45⁺, Ly6C⁻, MHC-II⁺, F4/80⁻, CD24⁺) in the CD45⁺ cell population in the B16F10-EV and B16F10-Rpso3 tumors by flow cytometry analysis (n=6 mice per group). (4E) Absolute numbers of tumor-infiltrating CD8⁺ cells (CD45⁺CD3⁺CD8⁺) in B16F10-EV and B16F10-Rspo3 tumors by flow cytometry analysis (n=5-6 mice per group). (4F) Representative flow plots (left) and summary (right) of the percentages of tumor-infiltrating CD8⁺ cells expressing Granzyme B, perforin, and CD69 in the B16F10-EV and B16F10-Rpso3 tumors (n=4-8 mice per group). (4G) Percentages of tumor-infiltrating NK cells and CD8⁺ T cells in the CD45⁺ cell population by flow cytometry analysis of Pan02-EV and Pan02-Rspo3 tumors (n=9-11 mice per group). (4H) Representative flow plots (left) and summary (right) of the percentages of Granzyme B-expressing tumor-infiltrating NK cells and CD8⁺ T cells in the Pan02-EV and Pan02-Rpso3 tumors (n=9-11 mice per group). (4I) Growth curves of B16F10-EV and B16F10-Rspo3 cells in syngeneic B6 mice treated with isotype control, anti-NK1.1 depletion antibody, anti-CD8a depletion antibody, or both (n=5-8 mice per group). (4J) Growth curves of B16F10-EV and B16F10-Rspo3 cells in Rag1^{-/-} mice (n=6 mice per group). (4K) Growth curves of Pan02-EV and Pan02-Rspo3 cells in syngeneic B6 mice treated with isotype control, anti-NK1.1 depletion antibody, anti-CD8a depletion antibody, or both (n=5-8 mice per group). (4L) Growth curves of Pan02-EV and Pan02-Rspo3 cells in Rag1^{-/-} mice (n=6 mice per group). Data are shown as mean±s.e.m. and are representative of at least two independent experiments. For 4A, 4B, and 4D-4H, Student's t-tests or Welch's t-tests were performed. For 4I-4L, two-way ANOVA tests were performed. P<0.05 is considered as statistically significant. ns, not statistically significant, *P<0.05, **P<0.01, ***P<0.001.

FIG. 5A-5G. R-spondin3 promotes MYC expression in NK cells in the TME. (5A) Quantitative RT-PCR results of Wnt target genes of flow-sorted CD11b⁺CD27⁻ NK cells from tumor tissues (n=4 for each group). (5B) Flow analysis of tumor-infiltrating NK cells from B16F10-EV or B16F10-Rspo3 tumors inoculated subcutaneously to Myc^{G/G} mice (n=5 for each group). Median fluorescence intensities of GFP for the indicated NK cell subpopulations are shown. (5C-5D) Quantitative RT-PCR results of rRNA (5C) or ribosomal protein mRNA (5D) of flow-sorted tumor-infiltrating NK cells from B16F10-EV or B16F10-Rspo3 tumor tissues. Data are shown as mean±s.d. Expression levels were normalized by cell numbers sorted. (5E) Median FSC intensities of flow analyzed tumor-infiltrating NK cells (CD45⁺CD3-NK1.1⁺DX5⁺) from B16F10-EV and B16F10-Rspo3 tumors (n=4-5 per group). (5F) Tumor growth curves of B16F10-EV and B16F10-Rspo3 in Ncr1^{Cre} and Myc^{Δ/Δ}/Ncr1^{Cre} mice (n=4-5 per group). (5G) Tumor growth curves of B16F10-EV and B16F10-Rspo3 cells in Ncr1^{Cre} and Myc^{Δ/Δ}/Ncr1^{Cre} mice depleted with NK cells (left) or CD8⁺ T cells (right) (n=4-5 per group). For 5A-5D, and 5F-5G, two-way ANOVA with or without Sidak's multiple comparisons tests were performed. For 5E, student's t-test was performed. Data represent at least two independent experiments. Data are shown as mean±s.e.m. unless otherwise noted. P<0.05 is considered as statistically significant. ns, not significant, *P<0.05, **P<0.01, ***P<0.001 and ****P<0.0001.

FIG. 6A-6J. R-spondin3 sensitizes tumors to PD-1 blocking therapy. (6A) Experimental design for panels B-E. B16F10-EV or B16F10-Rspo3 cells (5×10⁵ cells) were inoculated subcutaneously to B6 mice. 200 ug anti-PD1 antibody or isotype antibody were intraperitoneally injected at days 8, 11, and 14. (6B) Tumor growth curves of B16F10-EV and B16F10-Rspo3 cells with isotype or anti-PD1 therapy (n=6-8 mice per group). (6C) Representative tumor pictures (left) and summary of tumor weights (right) of B16F10-EV and B16F10-Rspo3 tumors with isotype or anti-PD1 therapy dissected 18 days after inoculation. (6D) Survival curves and results of the Log-rank test of B16F10-EV and B16F10-Rspo3 tumors with isotype or anti-PD1 therapy (n=6-8 mice for each group). (6E) Absolute numbers of tumor-infiltrating NK cells (CD45⁺CD3-NK1.1⁺DX5⁺) and CD8⁺ cells (CD45⁺CD3⁺CD8⁺) in B16F10-EV and B16F10-Rspo3 tumors with isotype or anti-PD1 antibody therapy (n=3-6 mice per group). (6F) Experimental design for panels G-H. Pan02-EV or Pan02-Rspo3 cells (5×10⁵ cells) were inoculated subcutaneously to B6 mice. 200 ug anti-PD1 antibody or isotype antibody were intraperitoneally injected at days 8, 11, 14 and 17. (6G) Tumor growth curves of Pan02-EV and Pan02-Rspo3 cells with isotype or anti-PD1 therapy (n=6-8 mice per group). (6H) Survival curves and results of the Log-rank test of B16F10-EV and B16F10-Rspo3 tumors with isotype or anti-PD1 therapy (n=6-8 mice for each group). (6I) Growth curves of B16F10-EV and B16F10-Rspo3 cells with or without anti-PD1 antibody therapy in mice treated with isotype control, anti-NK1.1 depletion antibody, or anti-CD8a depletion antibody (n=5-8 mice per group) were shown. (6J) Growth curves of Pan02-EV and Pan02-Rspo3 cells in mice treated with isotype control, anti-NK1.1 depletion antibody, or anti-CD8 depletion antibody (n=6-8 mice per group). For 6B, 6G, 6I, and 6J, two-way ANOVA tests were performed. For 6C and 6E, two-way ANOVA tests with Tukey's multiple comparisons were performed. Data are representative of at least two independent experiments. Data are shown as mean±s.e.m. P<0.05 is considered as statistically significant. ns, not statistically significant, *P<0.05, **P<0.01, and ***P<0.001.

FIG. 7A-7G. Correlation of RSPO genes with NK-cell signature in tumor tissues and normal tissues. (7A-7C) Results of Spearman's rank correlation analyses of RSPO1 (7A), RSPO2 (7B), RSPO4 (7C) with NK-cell signature genes using TCGA datasets are plotted with coefficient R and −log₁₀ (P-value). Cancer types with P-value<0.01 and R>0.45 are marked red. (7D-7E) Correlation of RSPO3 (7D) or RSPO1 (7E) in TCGA tumor tissues and normal tissues. P-values and Spearman's rank correlation coefficients are shown. (7F) Overall comparison of Spearman's rank correlation coefficients for RSPO1 with NK-cell signature for a total of 19 types of tumors and normal tissues. Wilcoxon tests were performed. (7G) Results of multivariant regression analysis of NK-cell signature with the indicated factors using TCGA datasets of SKCM and PAAD. Abbreviations for the cancer types and detailed summary are listed in Table 1.

FIG. 8A-8F. RSPO3 and RSPO1 correlate with anti-tumor immune cell signatures and better prognosis in multiple cancers. (8A-8B) Results of Spearman's rank correlation analyses of cDC1 signature (8A) and T-cell signature (8B) with RSPO3 using TCGA datasets are plotted with coefficient R and −log₁₀ (P-value). Cancer types with P-value<0.01 and R>0.45 are marked red. (8C) Spearman's rank correlation plots for RSPO3 with CD69, GZMA, GZMB, and IFNG in CHOL, BRCA and LUSC of TCGA datasets. (8D) Spearman's rank correlation plots for RSPO1 with CD69, GZMA, GZMB, and IFNG in CHOL and PAAD of TCGA datasets. (8E-8F) Kaplan-Meier curves showing the prognostic impact of RSPO3 (8E) and RSPO1 (8F) expression levels for the overall survival of patients with the indicated cancer types in TCGA datasets. Hazard ratios (HR) and P values of Log-rank tests are shown. Patients were grouped by the median expression levels. Abbreviations for the cancer types are listed in Table 1.

FIG. 9A-9B. Mouse Lgr gene expressions. (9A) Heatmap visualization of the gene expression levels of Lgr4, Lgr5, Lgr6 in mouse spleen NK cell subsets by immGen project. (9B) The expression of Lgr4, Lgr5, Lgr6, Ncr1, and Klrb1c in Haemopedia RNA-seq datasets.

FIG. 10A-10L. Exogenous expression of R-spondin3 in the TME inhibits tumor progression. (10A) Quantitative RT-PCR analysis of the mRNA levels of Rspo3 in different murine cancer cell lines. B16F10 (melanoma), LLC1 (lung Lewis carcinoma), MC38 (colon adenocarcinoma). AT3 (mammary carcinoma), Pan02 (pancreatic carcinoma). Expression values are normalized to B16F10 cells. (10B) Quantitative RT-PCR analysis of the mRNA levels of Rspo3 in B16F10-EV and B16F10-Rspo3 cells. Expression values are normalized to B16F10-EV cells. (10C) Protein expression of R-spondin3 in B16F10-EV and B16F10-Rspo3 cells. Quantifications of band intensities are shown. (10D) In vitro growth curves of B16F10-EV and B16F10-Rspo3 cells. (10E-10F) Summary of tumor weights of B16F10-EV and B16F10-Rspo3 cells inoculated to NRG mice (10E) or syngeneic B6 mice (10F) and dissected 18 days after inoculation (n=7-8 mice per group). (10G) Quantitative RT-PCR analysis of the mRNA levels of Rspo3 in Pan02-EV and Pan02-Rspo3 cells. Expression values are normalized to Pan02-EV cells. (10H) Protein expression of R-spondin3 in Pan02-EV and Pan02-Rspo3 cells. Quantifications of band intensity are shown. (10I) In vitro growth curves of Pan02-EV and Pan02-Rspo3 cells. (10J-10K) Summary of tumor weights of Pan02-EV and Pan02-Rspo3 cells inoculated to NRG mice (10E) or syngeneic B6 mice (10F) and dissected 33 days after inoculation (n=6-7 mice per group). (10L) Growth curves of B16F10-EV and B16F10-Rspo3 cells in Lgr6-1 mice (n=5-7 mice per group). Data are representative of at least two independent experiments. Data are shown as mean±s.e.m. For panels D, I, and L, two-way ANOVA tests were performed. For 10E-10F and 10J-10K, student's t-tests were performed. P<0.05 is considered as statistically significant. ns, not significant, *P<0.05, **P<0.01, ***P<0.001 and ****P<0.0001.

FIG. 11A-11K. Exogenous expression of R-spondin3 in the TME enhances anti-tumor immunity. (11A) Absolute numbers of tumor-infiltrating CD45+ cells in B16F10-EV and B16F10-Rspo3 tumors (n=5-6 mice per group) by flow cytometry analysis. (11B) Percentages of NK cells (CD45⁺CD3-NK1.1⁺DX5⁺) in the CD45⁺ cell population of B16F10-EV and B16F10-Rspo3 tumors by flow cytometry analysis (n=5-6 mice per group). (11C) Immunohistochemical staining by anti-NK1.1 antibody in B16F10-EV and B16F10-Rspo3 tumor tissues. Representative pictures (left) and frequency of positively stained cells (right) are shown. Pictures are shown as 200×. (11D) Cytotoxicity of freshly isolated tumor-infiltrating NK cells from B16F10-EV and B16F10-Rspo3 tumors was measured with chromium (51Cr)-release assays using B16F10 cells as target cells with the indicated E: T (effector: target ratios). Purified NK cells were pooled from four mice per group. (11E) Representative flow plots (left) and percentages of CD103⁺ DC (lineage [CD90.2, CD45R, Ly6G, NK1.1]⁻, CD45⁺, Ly6C-, MHC-II⁺, F4/80⁻, CD24⁺) (right) in the MHC-II⁺ cell population by flow cytometry analysis in the B16F10-EV and B16F10-Rpso3 tumors (n=6 mice per group). (11F) Percentages of CD8⁺ T cells (CD45⁺CD3-CD8⁺) in the CD45⁺ cell population of B16F10-EV and B16F10-Rspo3 tumors by flow cytometry analysis (n=7-9 mice per group). (11G) Immunohistochemical staining by anti-CD8 antibody in B16F10-EV and B16F10-Rspo3 tumor tissues. Representative pictures (left) and frequency of positively stained cells (right) are shown. Pictures are shown as 200×. (11H) Representative flow plots (left) and summary (right) of the percentages of IFN-γ-expressing tumor-infiltrating CD8+ cells in the B16F10-EV and B16F10-Rpso3 tumors (n=4 mice per group). (11I-11J) Absolute numbers of tumor-infiltrating CD45⁺ cells (11I), NK cells and CD8⁺ T cells (11J) in Pan02-EV and Pan02-Rspo3 tumors (n=8 mice per group) by flow cytometry analysis. (11K) Representative pictures (left) and scoring of stromal areas (right) of the H&E staining of Pan02-EV and Pan02-Rspo3 tumor tissues. Data represent at least two independent experiments and are shown as mean±s.e.m. Statistical analyses were performed using student's t-test or Welch's t-test. P<0.05 is considered as statistically significant. ns, not significant, *P<0.05, **P<0.01, ***P<0.001 and ****P<0.0001.

FIG. 12A-12H. Depletion of CD8⁺ T and NK cells in tumor-bearing mice. (12A) Depletion experimental scheme in B16F10 tumor models. (12B-12C) Verification of depletion. Representative flow plots of CD8⁺ T cells and NK cells in the peripheral blood (12B) and tumor tissues (12C) of mice bearing B16F10 tumors treated with isotype control antibody, anti-CD8a antibody, or anti-NK1.1 antibody. Samples were obtained on day 17 after tumor inoculation. Data are representative of at least two independent experiments. (12D) Survival curves and results of the Log-rank tests of B16F10-EV and B16F10-Rspo3 tumors treated with isotype control, anti-NK1.1 depletion antibody, anti-CD8a depletion antibody, or both depletion antibodies (n=5-8 mice per group). (12E) Survival curves and result of the Log-rank test of B16F10-EV and B16F10-Rspo3 tumors in Rag1^{-/-} mice (n=6 mice for each group). (12F) Depletion experimental scheme in Pan02 tumor models. (12G-12H) Verification of depletion. Representative flow plots of CD8⁺ T cells and NK cells in the peripheral blood (12G) and tumor tissues (12H) of mice bearing Pan02 tumors treated with isotype control antibody, anti-CD8a antibody, or anti-NK1.1 antibody. Samples were obtained on day 17 after tumor inoculation. Data are representative of at least two independent experiments.

FIG. 13A-13B. Reduced ribosomal biogenesis in NK cells with MYC deficiency. CD3⁻NK1.1⁺DX5⁺NK cells sorted by flow cytometry from the splenic cells of three Myc^{f/f} and two Myc^{Δ/Δ}/Ncr1^{Cre} mice were submitted for RNA-seq analysis. (13A) Result of KEGG pathway enrichment analysis performed for the 65 down-regulated genes (FDR<0.05) (Not shown). (13B) Gene set enrichment analysis of translation-associated genes.

FIG. 14A-14D. R-spondin3 sensitizes tumors to PD-1 blocking therapy. (14A) Growth curves of B16F10-EV (left) and B16F10-Rspo3 (right) tumors in individual syngeneic B6 mice (n=5-6 mice per group). (14B) Response rates of B16F10-EV and B16F10-Rspo3 tumors to anti-PD1 therapy. Statistical significance is performed with Fisher's exact test. (14C) Immunohistochemical staining by anti-NK1.1 and anti-CD8 antibodies in B16F10-EV and B16F10-Rspo3 tumors with isotype or anti-PD1 antibody therapy. Representative pictures (left) and frequency of positively stained cells (right) are shown. Pictures are shown as 200×. (14D) Tumor growth curves of B16F10-Rspo3 tumors with isotype or anti-PD1 antibody therapy in syngeneic B6 mice treated with isotype or anti-NK1.1 depletion antibody (n=6-8 mice per group).

FIG. 15. Working model: R-spondin3 enhances anti-tumor immunity and affects cancer outcomes. Left: Tumor containment. Endothelial cells and cancer-associated fibroblasts (CAF) secreted R-spondin3 potentiates the Wnt signaling in anti-tumor immune cells, including NK cells, through LGR6-dependent or -independent mechanism. Endocytosis of the R-spondin-LGR-ZNRF3/RNF43 complex leads to membrane clearance of the E3 ligases and persistence of Wnt receptors Frizzled (Fz) on the cell surface, thereby promoting Wnt signaling strength and duration. Whether G-protein mediated signaling by LGR is involved or not in the biological processes is unspecified. Enhanced MYC expression and ribosomal biogenesis gene expressions ensure the prompt translation of the mRNA pool in NK cells enabling effective cytotoxicity upon encountering tumor cells. Meanwhile, activated NK cells interact with other cells in the TME, including dendritic cells and T cells, to further elicit the adaptive anti-tumor immunity to contain the tumor. Right: Tumor progression. Tumor cells with intrinsic Wnt signaling activation secret a large amount of DKK1 to the TME, inhibiting the Wnt signaling activities of the surrounding cells, which include NK cells. R-spondins can potently counteract DKK1-mediated Wnt signaling inhibition. However, multiple factors in the TME may contribute to an insufficient level of R-spondin3 in TME. The subsequently impaired anti-tumor immunity results in tumor progression.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

“Sequence identity” as used herein indicates a nucleic acid sequence that has the same nucleic acid sequence as a reference sequence, or has a specified percentage of nucleotides that are the same at the corresponding location within a reference sequence when the two sequences are optimally aligned. For example a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

Natural killer (NK) cells and T cells are key effectors of anti-tumor immune responses and major targets of checkpoint inhibitors. In multiple cancer types, Applicant found that the expression of Wnt signaling potentiator R-spondin genes (e.g. RSPO3) is associated with favorable prognosis and is positively correlated with gene signatures of both NK cells and T cells. While endothelial cells and cancer-associated fibroblasts comprise the R-spondin3-producing cells, NK cells and T cells correspondingly express the R-spondin3 receptor LRG6 within the tumor microenvironment. Exogenous expression or intratumor injection of R-spondin3 in tumors enhanced the infiltration and function of cytotoxic effector cells, which led to tumor regression. NK cells and CD8⁺ T cells independently and cooperatively contributed to R-spondin3-induced control of distinct tumor types. The effect of R-spondin3 was mediated in part through upregulation of MYC and ribosomal biogenesis. R-spondin3 expression enhanced tumor sensitivity to anti-PD1 therapy, thereby highlighting new therapeutic avenues.

Applicant's study identifies novel targets in enhancing anti-tumor immunity and sensitizing immune checkpoint inhibition, which provides a rationale for developing new immunotherapies against cancers. It also offers mechanistic insights on Wnt signaling-mediated modulation of anti-cancer immunity in the TME and implications for a putative R-spondin-LGR6 axis in regulating NK-cell biology.

In one aspect, a method for treating a cancer in an individual is disclosed. The method may comprise administering an R-spondin protein to said individual. Administration of an R-spondin protein may include administration of a variant of R-spondin, a fragment of R-spondin, or a precursor of R-spondin, such that the administration causes R-spondin activity to occur post-administration in the body of said individual. Administration may include any means of administration, including intra-tumor (direct) injection, intramuscular administration, intravenous administration, subcutaneous administration, or combinations thereof. The R-spondin may be administered in a composition comprising a suitable, sterile carrier, comprising one or more carrier materials or pharmaceutically acceptable excipients as described herein.

R-Spondin. The terms “R-spondin” and “RSPO” refer to a native R-spondin from any vertebrate source, including mammals such as primates (e.g., humans) and rodents (e.g., mice and rats), unless otherwise indicated. The term encompasses “full-length,” unprocessed R-spondin as well as any form of R-spondin that results from processing in the cell. The term also encompasses naturally occurring variants of R-spondin, e.g., splice variants or allelic variants. R-spondin is a family of four proteins, R-spondin 1 (RSPO1), R-spondin 2 (RSPO2), R-spondin 3 (RSPO3), and R-spondin 4 (RSPO4). Accession numbers for the R-spondins contemplated herein are as follows: RSPO1, ID: 284654; RSPO2, ID: 340419; RSPO3, ID: 84870; RSPO4, ID: 343637. “R-Spondin variant,” “RSPO variant,” or variations thereof, means an R-spondin polypeptide or polynucleotide, generally being or encoding an active R-Spondin polypeptide, as defined herein having at least about 80% amino acid sequence identity with any of the R-Spondin as disclosed herein. Such R-Spondin variants may include, for instance, R-Spondin wherein one or more nucleic acid or amino acid residues are added or deleted. An R-spondin variant may have at least about 80% sequence identity, alternatively at least about 81%, 82%, 83%, 84%, 85%, 86%, 87%, 88%, 89%, 90%, 91%, 92%, 93%, 94%, 95%, 96%, 97%, 98%, or 99% sequence identity, to R-Spondin as disclosed herein. R-Spondin variant may be at least about 10 residues in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 in length, or more. Optionally, R-Spondin variant may have or encode a sequence having no more than one conservative amino acid substitution as compared to R-Spondin, alternatively no more than 2, 3, 4, 5, 6, 7, 8, 9, or 10 conservative amino acid substitution as compared to R-Spondin. In one aspect, the R-spondin protein may be a fragment of a full-length R-spondin protein.

In one aspect, the R-spondin may be a fragment or a variant, having a furin-1 domain furin-1 domain is known to be able to bind to ZNRF3/RNF43 (part of Wnt receptor complex) with low affinity. The high-affinity binding to LGR or HSPG may be a bait like mechanism to facilitate furin-1 binding to ZNRF3/RNF43, the ultimate working component in driving the enhanced Wnt signaling. In one aspect, the fragment or variant of R-spondin may include a furin-1 domain and a furin-2 domain. In one aspect, the fragment or variant of R-spondin may include a furin-1 domain, and thrombospondin type 1 (TSP) and basic region (BR) (TSP/BR). In a further aspect, the fragment or variant of R-spondin may include a furin-1 domain, a furin-2 domain, and a thrombospondin type 1 (TSP) domain and a basic region (BR) (TSP/BR) domain.

In one aspect, the fragment or variant of R-spondin may be one which binds to Leucine-rich repeat-containing G-protein coupled receptor 4 (LGR4), Leucine-rich repeat-containing G-protein coupled receptor 5 (LGR5), and/or Leucine-rich repeat-containing G-protein coupled receptor 6 (LGR6).

In one aspect, the R-spondin protein, including a variant or a fragment thereof, may be administered via an R-spondin precursor. The R-spondin precursor may comprise DNA, RNA or a combination thereof, wherein the precursor may be used to express R-spondin in vivo after administration to the individual. In one aspect, the R-spondin precursor may further comprise an expression vector operably linked to an R-spondin gene or gene fragment, which may include a variant thereof, wherein said expression vector expresses the R-spondin or gene fragment (or variant thereof) in vivo.

In one aspect, R-spondin may be administered in an amount of from about 5 μg/kg to about 500 μg/kg, or from about 10 μg/kg to about 250 μg/kg, or from about 25 μg/kg to about 100 μg/kg, about 50 μg/kg tumor weight. In certain aspects, the R-spondin may be administered in a sterile saline solution or in a carrier as described herein. The R-spondin may be administered at an interval selected from daily, every two days, every three days, every four days, every five days, every six days, every seven days, every two weeks, every three weeks, and monthly.

The method may be employed to treat an individual having a cancer. Such cancers may include leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In one aspect, the cancer may be a cancer that presents as a solid tumor, such as a sarcoma, carcinoma, or lymphoma, comprising the step of administering a disclosed compound, or a pharmaceutically acceptable salt thereof, to a patient in need thereof. The term “solid tumor” refers to malignancies/cancers formed of abnormal masses of tissue that usually do not contain cysts or liquid areas. Solid tumors are named/classified according to the tissue/cells of origin. Examples include sarcomas and carcinomas. In some embodiments, the cancer may be selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In one aspect, the administering may improve a prognosis in an individual diagnosed with a cancer, for example a cancer selected from Skin Cutaneous Melanoma (SKCM), pancreatic adenocarcinoma (PAAD), lung squamous cell carcinoma (LUSC), and head and neck squamous carcinoma (HNSC), breast invasive carcinoma (BRCA), and cholangiocarcinoma (CHOL) BRCA: Breast invasive carcinoma, Thyroid carcinoma (THCA), Bladder Urothelial Carcinoma (BLCA), Colon adenocarcinoma (COAD), or Uveal Melanoma (UVM). The administering may be to the site of the cancer, such as via direct injection to the solid tumor. In further aspect, the administration may be via intravenous injection, which may further include intra-tumor injection, which may occur simultaneously, or in sequence.

The methods may further employ the administration of an immune checkpoint inhibitor. The checkpoint inhibitor may be administered at a time selected from prior to said R-spondin administration, during said R-spondin administration, after said R-spondin administration, or a combination thereof. The immune checkpoint inhibitor may be selected from a Programmed cell death protein 1 (PD-1) or Programmed death-ligand 1 (PD-L1) inhibitor. For example, the Programmed death-ligand 1 (PD-1) inhibitor may be selected from nivolumab, pembrolizumab, cemiplimab, and combinations thereof. In one aspect, the Programmed death-ligand 1 (PD-L1) inhibitor may be administered and may be selected from atezolizumab, avelumab, durvalumab, and combinations thereof. In further aspects, the method may comprise administering a cytotoxic T-lymphocyte-associated protein 4 (CTLA-4) inhibitor, for example a CTLA-4 inhibitor selected from ipilimumab, tremelimumab, and combinations thereof. In further aspects, the method may comprise administering a checkpoint inhibitor that targets one or more of LAG-3, TIM-3, TIGIT, VISTA, B7-H3, and BTLA.

In one aspect, a method of treating an individual having a cancer using a diagnostic method is disclosed. In this aspect, the method may comprise determining an expression level of R-spondin in an individual diagnosed with a cancer; and administering an R-spondin protein or precursor thereof to said individual having a decrease in R-spondin level as compared to a normal level. In one aspect, the R-spondin protein may be selected from R-spondin1, R-spondin2, R-spondin3, R-spondin4, and combinations thereof. The R-spondin and administration thereof may be as described above. The determining may comprise any method of detecting expression of one or more R-Spondin protein(s), including determining an intra-tumor expression level, determining a circulating expression level (such as in the blood, or as measured in serum or plasma or any body fluid that allows for detection of expression of R-spondin), and/or combinations thereof. In this aspect, the method may include a comparison step, in which the measured level of R-spondin expression is compared to a control value, such as an average level of R-spondin expression as determined in a healthy individual, the term “healthy” referring to an individual not having a cancer expected to cause elevated R-spondin expression. Such determining of a control value will be readily understood by one of ordinary skill in the art.

Pharmaceutical Compositions

In one aspect, active agents provided herein may be administered in an dosage form selected from intratumor, intravenous or subcutaneous unit dosage form, oral, parenteral, intratumor, intravenous, and subcutaneous. In some embodiments, active agents provided herein may be formulated into liquid preparations for, e.g., intratumor administration, intravenous administration, subcutaneous administration, and/or oral administration. In some aspects, the compositions may be in a unit dosage forms configured for administration once a day, twice a day, or more.

In one aspect, pharmaceutical compositions are isotonic with the blood or other body fluid of the recipient. The compositions may be formulated with an isotonic agent. As used herein, the term “isotonic agent” refers to a component that functions to partially maintain isotonicity of the formulation and the protein level, and partially maintain the level, ratio, or proportion of the therapeutically active protein/fragment/variant and/or precursor present in the formulation. The isotonic agent may be used to maintain the same osmotic pressure as blood plasma, and so can be intravenously injected into a subject without changing the osmotic pressure of the subject's blood plasma. Osmotic pressure may be suitable for injection of the formulation. Isotonicity may be attained using sodium tartrate, propylene glycol or other inorganic or organic solutes, for example, sodium chloride. Buffering agents may be employed, such as acetic acid and salts, citric acid and salts, boric acid and salts, and phosphoric acid and salts. Parenteral vehicles may include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Viscosity of the pharmaceutical compositions may be maintained at the selected level using a pharmaceutically acceptable thickening agent. Methylcellulose is useful because it is readily and economically available and is easy to work with. Other suitable thickening agents include, for example, xanthan gum, carboxymethyl cellulose, hydroxypropyl cellulose, carbomer, and the like. In some embodiments, the concentration of the thickener will depend upon the thickening agent selected. An amount may be used that will achieve the selected viscosity. Viscous compositions are normally prepared from solutions by the addition of such thickening agents.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative is typically from about 0.02% to about 2% based on the total weight of the composition, although larger or smaller amounts may be desirable depending upon the agent selected. Reducing agents, as described above, may be advantageously used to maintain good shelf life of the formulation.

In one aspect, active agents provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and are thus chosen according to the intended application, such that the characteristics of the carrier are tailored to the selected route of administration.

The R-Spondin compositions may be provided in a variety of forms suited for the the type of administration, selected from, for example, aqueous or oil suspension, dispersible powder or granule, emulsion, hard or soft capsule, syrup or elixir, or tablet. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions. In some embodiments, an active agent provided herein may be administered by intravenous, parenteral, or other injection, in the form of a pyrogen-free, parenterally acceptable aqueous solution or oleaginous suspension. Suspensions may be formulated according to methods well known in the art using suitable dispersing or wetting agents and suspending agents. The preparation of acceptable aqueous solutions with suitable pH, isotonicity, stability, and the like, is within the skill in the art. In some embodiments, a pharmaceutical composition for injection may include an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils may be employed conventionally as a solvent or suspending medium. For this purpose, any bland fixed oil may be employed including synthetic mono or diglycerides. In addition, fatty acids such as oleic acid may likewise be used in the formation of injectable preparations. The pharmaceutical compositions may also contain stabilizers, preservatives, buffers, antioxidants, or other additives known to those of skill in the art. In aspects in which the administration is via injection, the duration of the injection may be adjusted depending upon various factors, and may comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

In some embodiments, active agents provided herein may additionally employ adjunct components conventionally found in pharmaceutical compositions in their art-established fashion and at their art-established levels. Thus, for example, the compositions may contain additional compatible pharmaceutically active materials for combination therapy or may contain materials useful in physically formulating various dosage forms, such as excipients, dyes, thickening agents, stabilizers, preservatives or antioxidants.

Formulations for oral use may also be provided as hard gelatin capsules, wherein the active ingredient(s) are mixed with an inert solid diluent, such as calcium carbonate, calcium phosphate, or kaolin, or as soft gelatin capsules. In soft capsules, the active agents may be dissolved or suspended in suitable liquids, such as water or an oil medium, such as peanut oil, olive oil, fatty oils, liquid paraffin, or liquid polyethylene glycols. Stabilizers and microspheres formulated for oral administration may also be used. Capsules may include push-fit capsules made of gelatin, as well as soft, sealed capsules made of gelatin and a plasticizer, such as glycerol or sorbitol. The push-fit capsules may contain the active ingredient in admixture with fillers such as lactose, binders such as starches, and/or lubricants, such as talc or magnesium stearate and, optionally, stabilizers.

Tablets may be uncoated or coated by known methods to delay disintegration and absorption in the gastrointestinal tract and thereby provide a sustained action over a longer period of time. For example, a time delay material such as glyceryl monostearate may be used. When administered in solid form, such as tablet form, the solid form typically comprises from about 0.001 wt. % or less to about 50 wt. % or more of active ingredient(s), for example, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets may contain the active ingredients in admixture with non-toxic pharmaceutically acceptable excipients including inert materials. For example, a tablet may be prepared by compression or molding, optionally, with one or more additional ingredients. Compressed tablets may be prepared by compressing in a suitable machine the active ingredients in a free-flowing form such as powder or granules, optionally mixed with a binder, lubricant, inert diluent, surface active or dispersing agent. Molded tablets may be made by molding, in a suitable machine, a mixture of the powdered active agent moistened with an inert liquid diluent.

In some embodiments, each tablet or capsule contains from about 1 mg or less to about 1,000 mg or more of a active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. In some embodiments, tablets or capsules are provided in a range of dosages to permit divided dosages to be administered. A dosage appropriate to the patient and the number of doses to be administered daily may thus be conveniently selected. In certain embodiments two or more of the therapeutic agents may be incorporated to be administered into a single tablet or other dosage form (e.g., in a combination therapy); however, in other embodiments the therapeutic agents may be provided in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents may be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, or karaya, or alginic acid or salts thereof. Binders may be used to form a hard tablet. Binders include materials from natural products such as acacia, starch and gelatin, methyl cellulose, ethyl cellulose, carboxymethyl cellulose, polyvinyl pyrrolidone, hydroxypropylmethyl cellulose, and the like. Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, may be included in tablet formulations.

Surfactants may also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations may be employed wherein the active agent or analog(s) thereof is incorporated into an inert matrix that permits release by either diffusion or leaching mechanisms. Slowly degenerating matrices may also be incorporated into the formulation. Other delivery systems may include timed release, delayed release, or sustained release delivery systems.

Coatings may be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments may be added for identification or to characterize different combinations of active agent doses.

When administered orally in liquid form, a liquid carrier such as water, petroleum, oils of animal or plant origin such as peanut oil, mineral oil, soybean oil, or sesame oil, or synthetic oils may be added to the active ingredient(s). Physiological saline solution, dextrose, or other saccharide solution, or glycols such as ethylene glycol, propylene glycol, or polyethylene glycol are also suitable liquid carriers. The pharmaceutical compositions may also be in the form of oil-in-water emulsions. The oily phase may be a vegetable oil, such as olive or arachis oil, a mineral oil such as liquid paraffin, or a mixture thereof. Suitable emulsifying agents include naturally-occurring gums such as gum acacia and gum tragamayth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

Pulmonary delivery of the active agent may also be employed. The active agent may be delivered to the lungs while inhaling and traverses across the lung epithelial lining to the blood stream. A wide range of mechanical devices designed for pulmonary delivery of therapeutic products may be employed, including but not limited to nebulizers, metered dose inhalers, and powder inhalers, all of which are familiar to those skilled in the art. These devices employ formulations suitable for the dispensing of active agent. Typically, each formulation is specific to the type of device employed and may involve the use of an appropriate propellant material, in addition to diluents, adjuvants, and/or carriers useful in therapy. The active ingredients may be prepared for pulmonary delivery in particulate form with an average particle size of from 0.1 um or less to 10 um or more, for example, from about 0.2, 0.3, 0.4, 0.5, 0.6, 0.7, 0.8, or 0.9 μm to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 μm. Pharmaceutically acceptable carriers for pulmonary delivery of active agent include carbohydrates such as trehalose, mannitol, xylitol, sucrose, lactose, and sorbitol. Other ingredients for use in formulations may include DPPC, DOPE, DSPC, and DOPC. Natural or synthetic surfactants may be used, including polyethylene glycol and dextrans, such as cyclodextran. Bile salts and other related enhancers, as well as cellulose and cellulose derivatives, and amino acids may also be used. Liposomes, microcapsules, microspheres, inclusion complexes, and other types of carriers may also be employed. Pharmaceutical formulations suitable for use with a nebulizer, either jet or ultrasonic, typically comprise the active agent dissolved or suspended in water at a concentration of about 0.01 or less to 100 mg or more of active agent per mL of solution, for example, from about 0.1, 1, 2, 3, 4, 5, 6, 7, 8, 9, or 10 mg to about 15, 20, 25, 30, 35, 40, 45, 50, 55, 60, 65, 70, 75, 80, 85, or 90 mg per mL of solution. The formulation may also include a buffer and a simple sugar (e.g., for protein stabilization and regulation of osmotic pressure). The nebulizer formulation may also contain a surfactant, to reduce or prevent surface induced aggregation of the active agent caused by atomization of the solution in forming the aerosol. Formulations for use with a metered-dose inhaler device generally comprise a finely divided powder containing the active ingredients suspended in a propellant with the aid of a surfactant. The propellant may include conventional propellants, such as chlorofluorocarbons, hydrochlorofluorocarbons, hydrofluorocarbons, and hydrocarbons. Example propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations for dispensing from a powder inhaler device typically comprise a finely divided dry powder containing active agent, optionally including a bulking agent, such as lactose, sorbitol, sucrose, mannitol, trehalose, or xylitol in an amount that facilitates dispersal of the powder from the device, typically from about 1 wt. % or less to 99 wt. % or more of the formulation, for example, from about 5, 10, 15, 20, 25, 30, 35, 40, 45, or 50 wt. % to about 55, 60, 65, 70, 75, 80, 85, or 90 wt. % of the formulation.

In some embodiments, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of a active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Natural killer (NK) cells are essential innate immune effector cells that can recognize and rapidly kill oncogenically transformed target cells. NK cell interactions with other immune cells in the tumor microenvironment (TME), such as dendritic cells (DC) and T cells, are crucial to magnify the overall immune response against the cancer^{[1,2]}. Supporting this notion, elevated numbers and enhanced functionality of NK cells are associated with better responses to immune checkpoint blockade therapies that largely target T cells^{[1]}, and emerging evidence indicates that direct targeting on NK cells may also exist^{[3,4]}. However, NK cells typically exhibit poor capacity to infiltrate tumors and frequently become functionally exhausted within tumors due to various immunosuppressive facets of the TME^{[5]}. Thus, the identification of molecular targets and development of therapeutic strategies to promote infiltration and maintain or restore anti-tumor functions of NK cells in the TME have been an outstanding clinical priority to improve cancer outcomes.

Wnt-signaling pathways control a wide range of cellular processes and are delicately regulated by a variety of positive or negative regulators with temporospatial specificity^{[6]}. Several recent studies highlight an role of Wnt signaling in regulating NK-cell anti-tumor functionality^{[7]}. Activation of the Wnt/β-catenin pathway via inhibition of GSK3β enhanced the maturation and function of NK cells^{[8]}. Cancer cell secretion of the Wnt signaling antagonists Dickkopf 1/2 (DKK1/2) facilitated the evasion of NK cell-mediated anti-tumor responses in certain contexts^{[9,10]}. On the other hand, hyperactivation of Wnt-β catenin pathway is often a hallmark of cancer cells and crucial in tumor formation. And evidence is also showing up for an immune cell exclusion phenotype associated with tumor cell-intrinsic aberrant β-catenin signaling activation across cancers^{[11,12]} Thus, the TME is believed to be controlled by an intricate interplay of Wnt agonists, antagonists, and anti-antagonists, and there could be certain components in the Wnt signaling pathway that play critical roles in tuning the activity and infiltration of NK cells in the TME.

The R-spondin gene family, RSPO1 thru RSPO4, encode four evolutionary conserved secreted proteins. R-spondins can potentiate canonical Wnt signaling in the low dose of Wnt following binding to the leucine-rich repeat-containing G-protein coupled receptors (LGR) LGR4, LGR5, and LGR6 with high affinity^{[13, 14].} Previous studies have described functions for R-spondins mainly in embryonic development, adult stem cell maintenance, and tumorigenesis^{[15,16].} However, the roles of R-spondins in modulating anti-tumor immunity remain ill-defined and largely unexplored.

LGR6 shares a similar structural basis with LGR4/5 and together they belong to the B-type LGR subfamily that is characterized by a long ectodomain containing 17 leucine-rich repeats (LRR)^{[16]}. These three LGRs were considered as obligate high-affinity receptors for R-spondins^{[13]}. While LGR-independent enhancement of Wnt signaling has also been reported recently for RSPO2 and RSPO3^{[17,18]}. LGR6 was shown to have a unique expression pattern and has been extensively reported to mark distinct types of adult stem cells in actively self-renewing tissues, such as epidermis and mammary glands^{[19-22]}. However, the expression and function of LGR6 in cell types other than the stem/progenitor cell populations remains unspecified.

Here Applicant identified R-spondin family members R-spondin3 and R-spondin1, which are mainly expressed by endothelial cells (EC) and cancer-associated fibroblasts (CAF) in the TME, as potential modulators of anti-tumor immunity in cancers. Exogenous expression of R-spondin3 in the TME promotes tumor suppression largely through NK cells, as well as CD8⁺ T cells. The mechanism of R-spondin3 enhancement of effector cell responses involves enhanced expression of the Wnt target gene MYC in NK cells. R-spondin3 and PD-1 blockade therapy cooperatively enhance immune control of tumors. These findings provide molecular and mechanistic insights on Wnt signaling components in modulating anti-tumor immunity and a strong rationale for developing novel anti-cancer immunotherapeutic strategies utilizing R-spondins.

Results:

RSPO3 and RSPO1 levels positively correlate with anti-cancer immune-cell signatures and better prognosis in multiple cancers.

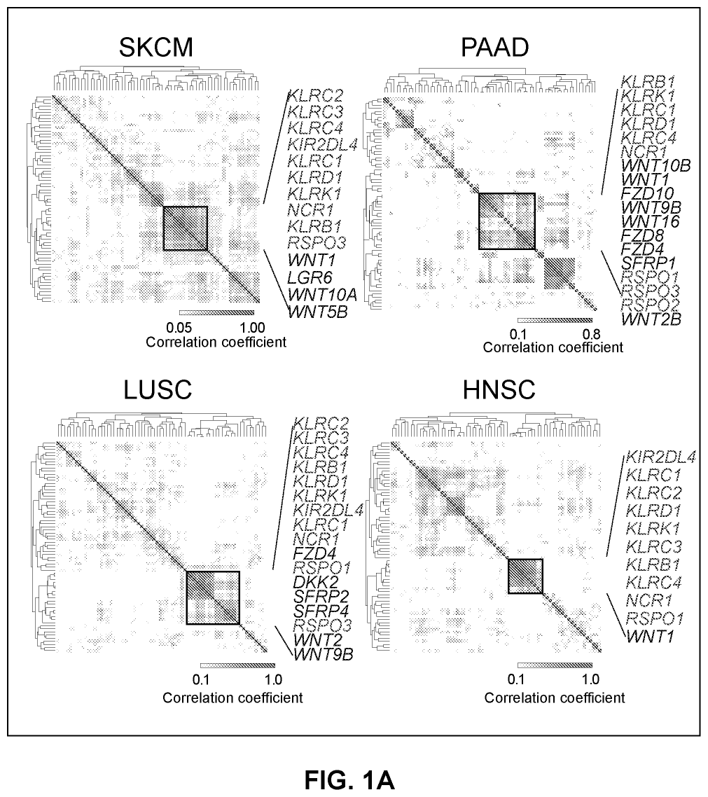

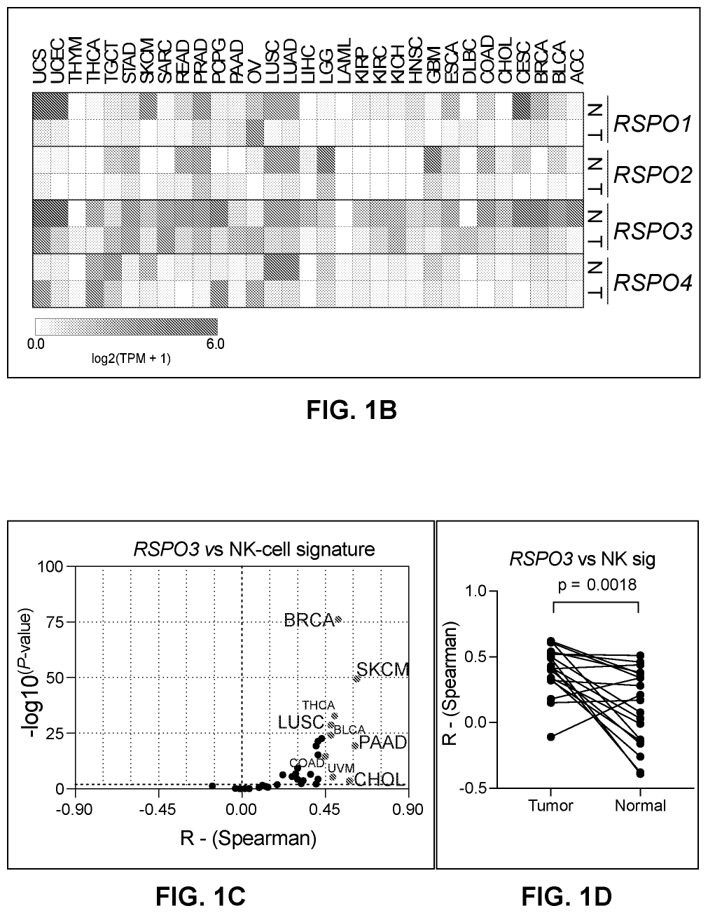

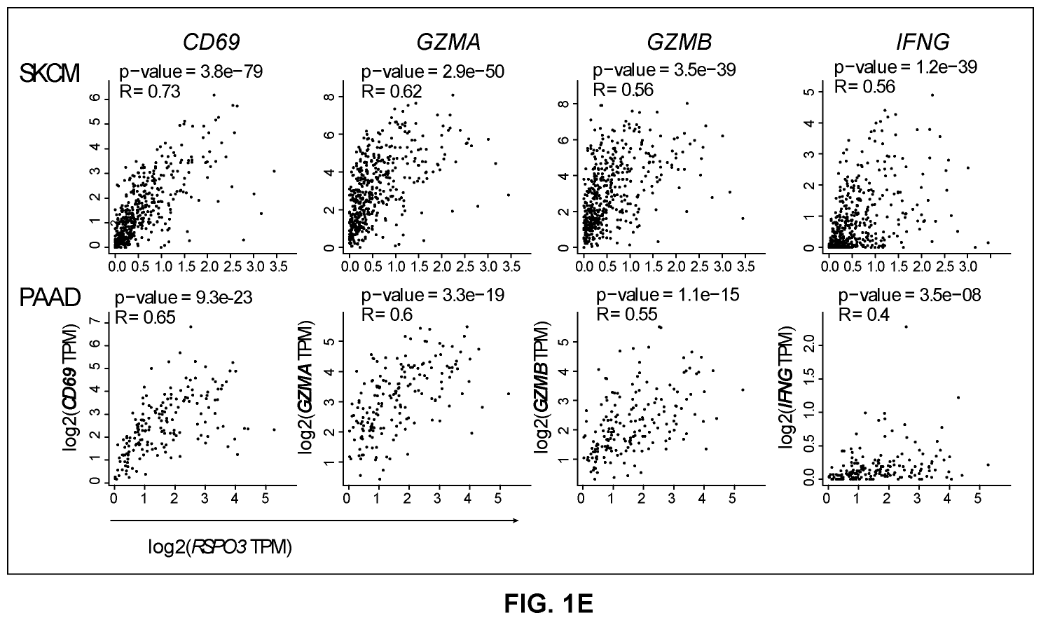

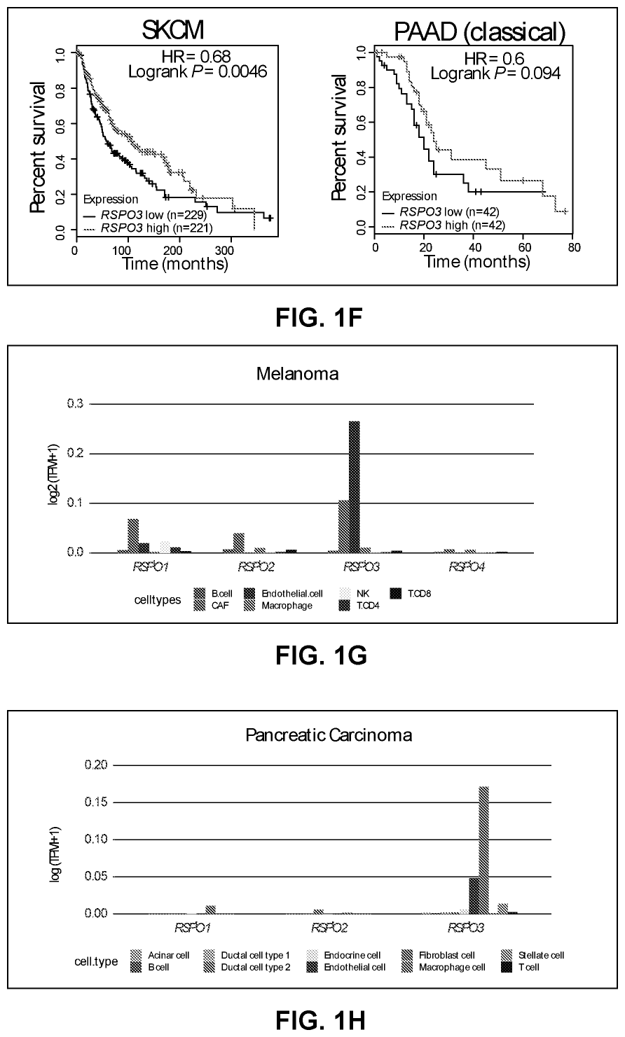

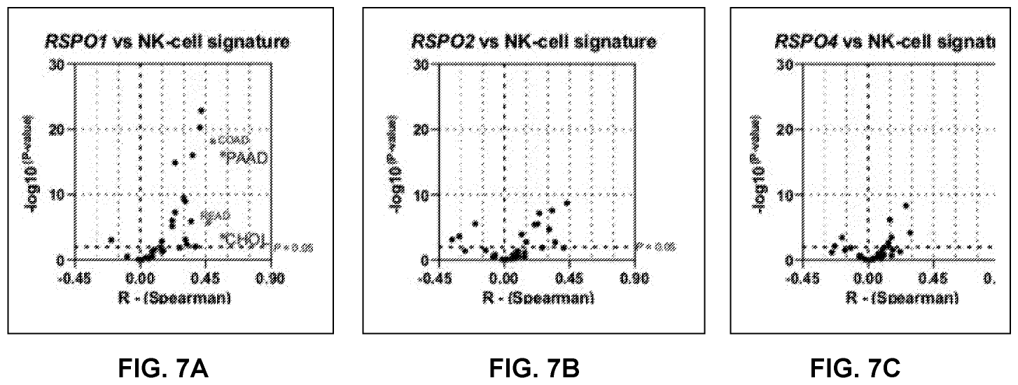

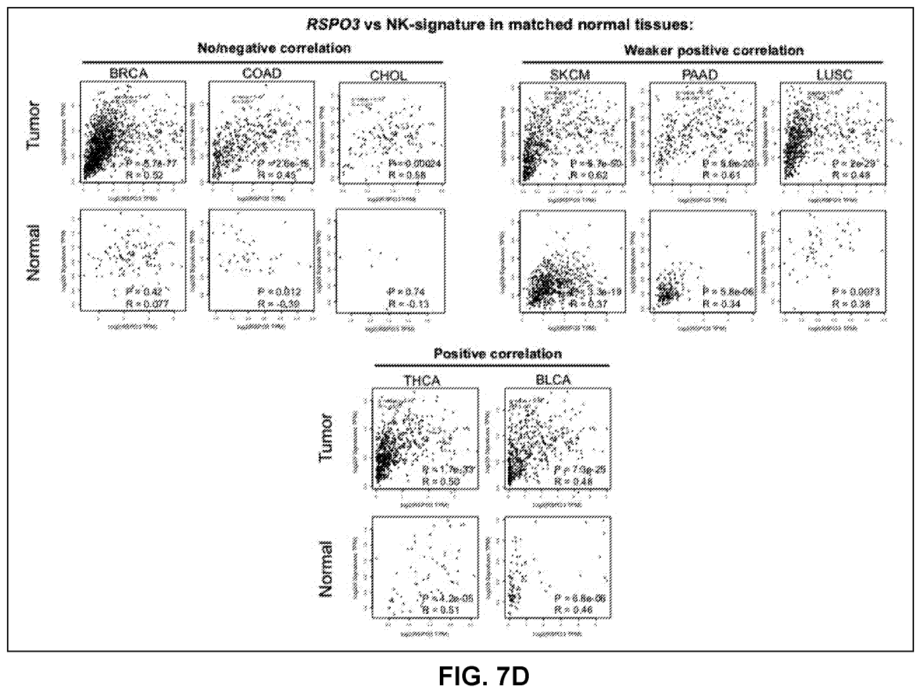

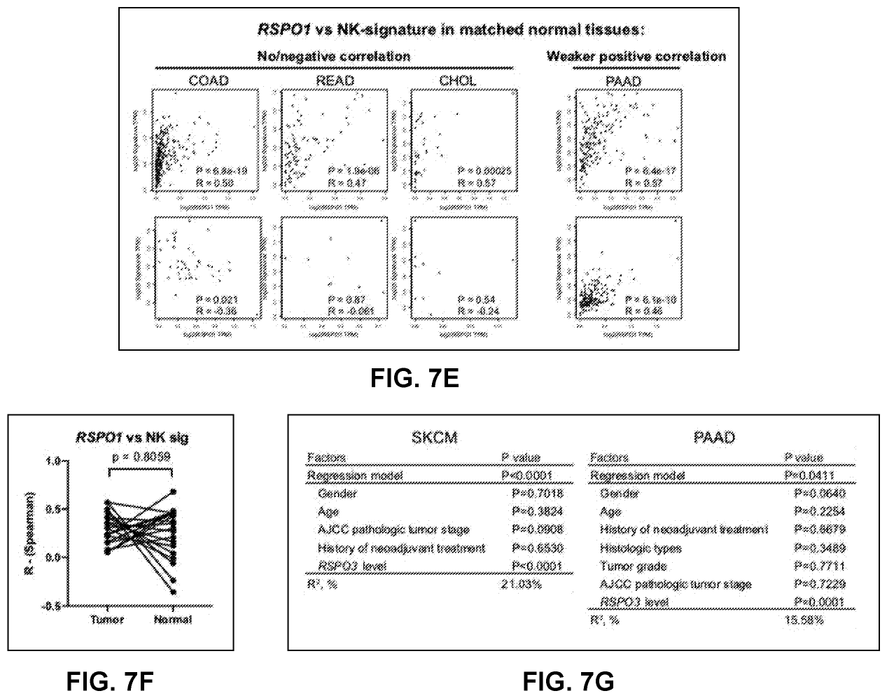

To explore Wnt signaling components that may regulate the anti-tumor immune responses of NK cells in the TME, Applicant generated correlation matrices with The Cancer Genome Atlas (TCGA) datasets using 60 genes encoding Wnt signaling components and 9 NK-cell signature genes (KIR2DL4, NCR1, KLRD1, KLRC1, KLRC2, KLRC3, KLRC4, KLRB1, KLRK1)^{[23]}. These genes are enriched in NK cells and can be used to indirectly infer the abundance of NK-cells within tumor tissue. RSPO1 and RSPO3 recurrently showed positive associations with the NK-cell signature genes in four cancer types: melanoma (SKCM), pancreatic adenocarcinoma (PAAD), lung squamous carcinoma (LUSC), and head and neck squamous carcinoma (HNSC) (FIG. 1, A). Notably, all four members of the R-spondin family are downregulated in the majority of cancer types compared to matched normal tissues (FIG. 1, B), suggesting a potential for a shared underlying mechanistic role of R-spondin in different cancers. Applicant then performed further correlation analyses specifically for the NK-cell signature genes with RSPO3 or RSPO1 in a total of 33 cancer types from TCGA datasets. Nine cancer types showed positive correlations (R>0.45, P<0.05) for RSPO3, with SKCM, PAAD, breast invasive carcinoma (BRCA), and cholangiocarcinoma (CHOL) showing strong correlations (R>0.5) (FIG. 1, C). Four cancer types showed positive correlations (R>0.45, P<0.05) for RSPO1, with PAAD and CHOL showing strong correlations (R>0.5) (FIG. 7, A). In contrast, RSPO2 or RSPO4 did not show positive correlations with the NK-cell signature in the TCGA datasets (R>0.45, P<0.05) (FIG. 7, B-C). Together, these data suggest positive correlations between the expression of RSPO3 or RSPO1 with NK-cell signature across different cancers, particularly strong in SKCM, PAAD, BRCA, CHOL, and LUSC.

To further investigate whether the correlations for RSPO3 or RSPO1 with NK-cell signature are unique in tumor tissues relative to normal tissues, Applicant analyzed the available TCGA normal tissues or The Genotype-Tissue Expression (GTEx) dataset. Results showed no or weaker positive correlations were observed in the counterpart normal tissues in the majority of cancer types analyzed, except for bladder urothelial carcinoma (BLCA) and thyroid carcinoma (THCA) (FIG. 7, D-E). A generally stronger correlation with NK-cell signature in tumor tissues relative to counterpart normal tissues was observed for RSPO3, while not for RSPO1 (FIG. 1, D, FIG. 7, F). The impact of confounding clinical factors on these association was assessed by multivariant analysis, including age, gender, history of neoadjuvant therapy, tumor grade, and tumor stage. The results showed that RSPO3 level remained as an independent factor that correlated with the expression of NK-cell signature in both SKCM and PAAD (FIG. 7, G). These data suggest a positive correlation between the expression of RSPO3 and NK-cell signature in tumor tissues.

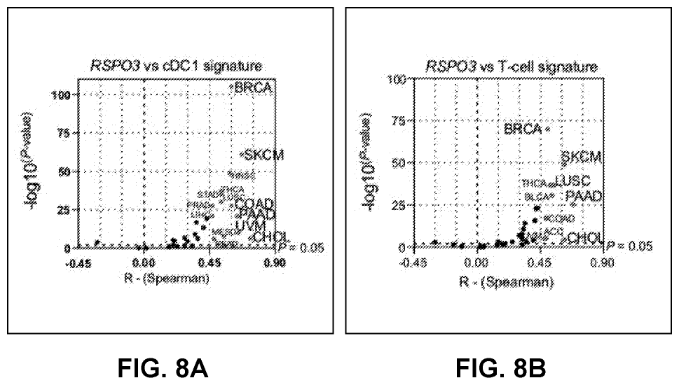

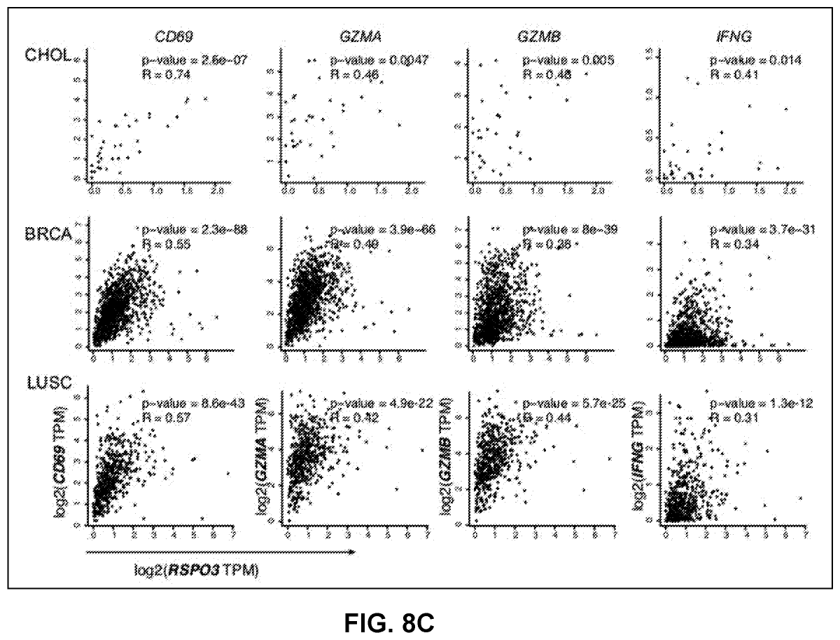

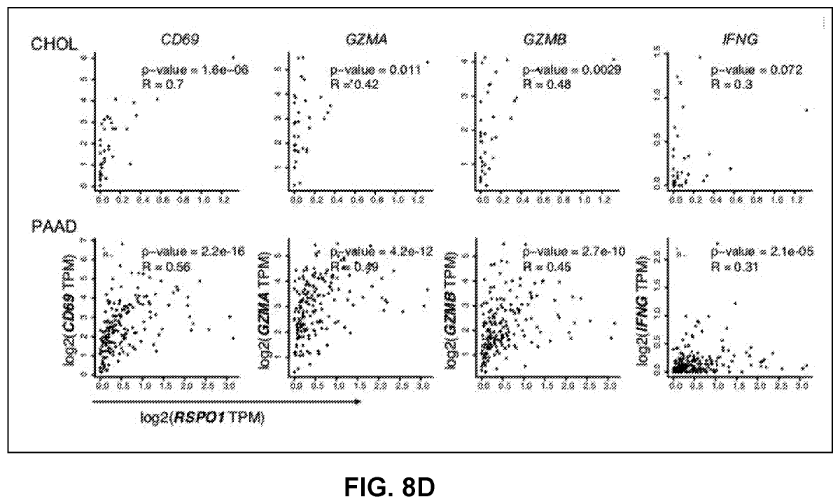

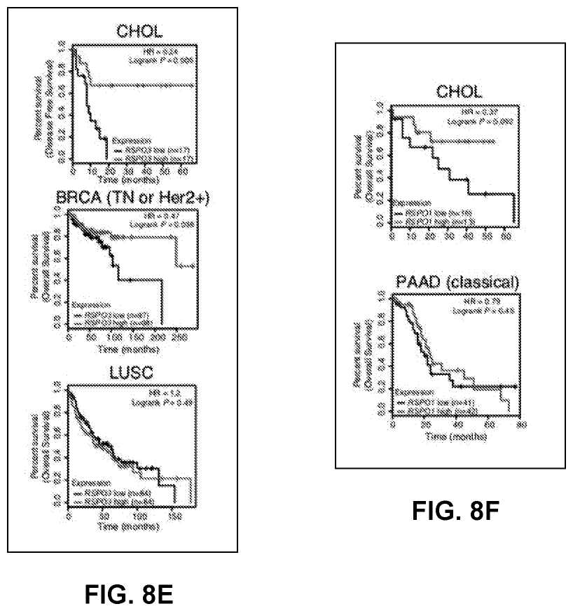

NK cells exhibit tight interactions with DC and T cells and subsequently affect the overall anti-cancer immune responses^{[1]}. To further explore whether the positive correlation seen for R-spondin genes with NK-cell signature could also be observed for DC or T-cell signature, Applicant performed correlation analyses for RSPO3 with a conventional type 1 dendritic cell (cDC1) signature (KIT, CCR7, BATF3, FLT3, ZBTB46, IRF8, BTLA, MYCL, CLEC9A, XCR1)^{[24]} and a T-cell signature (CD8A, CD8B, and CD3E). Fourteen cancer types showed positive correlations (R>0.45, P<0.05) between RSPO3 and DC1 signature, while 10 cancer types showed positive correlations between RSPO3 and T-cell gene signatures (FIG. 8, A-B). Applicant next checked whether RSPO3 or RSPO1 correlated with the expression of immune cell activation marker (CD69) or cytotoxic functional genes (GZMA, GZMB, IFNG) in the TME. Strong positive correlations (R>0.55, P<0.05) with RSPO3 were observed for CD69, GZMA, and GZMB in SKCM and PAAD (FIG. 1, E). Overall positive correlations (R=0.3-0.75, P<0.1) were observed for these markers with RSPO3 in CHOL, BRCA, and LUSC (FIG. 8, C) and with RSPO1 in PAAD and CHOL (FIG. 8, D). Together, these data suggest that high expression levels of RSPO3 or RSPO1 are associated with better anti-cancer immunity in the TME.

Survival analyses revealed that patients with higher levels of RSPO3 had a better prognosis in SKCM, PAAD (classical subtype), CHOL, and BRCA (non-luminal, triple-negative, Her2+) (FIG. 1, F, FIG. 8, E). Patients with higher levels of RSPO1 had better survival in CHOL (FIG. 8, F). Collectively, this body of evidence suggests that expression of RSPO3 or RSPO1 are associated with better NK and T cell activity as well as improved prognosis in cancers.

RSPO3 is expressed by ECs and CAFs in the TME.

To explore the source of R-spondins in the TME, Applicant analyzed available scRNAseq datasets of human melanoma and pancreatic carcinoma^{[25,26]}. Results showed that endothelial cells (ECs) and cancer-associated fibroblasts (CAFs) were the major cell populations that expressed RSPO1 and RSPO3 in the TME, with RSPO3 being much more abundantly expressed (FIG. 1, G-H). Low to negligible expression levels of RSPO2 and RSPO4 were observed in the TME of the two cancer types (FIG. 1, G-H). Further, immunohistochemistry staining of R-spondin3 and EC marker CD31 showed an abundant protein level of R-spondin3 in the melanoma TME with regional overlap with CD31 staining (FIG. 1I). These data indicate that EC and CAF are among the major cell sources of R-spondin3 in the TME.

R-spondin receptor LGR6 is prominently expressed by human NK cells.

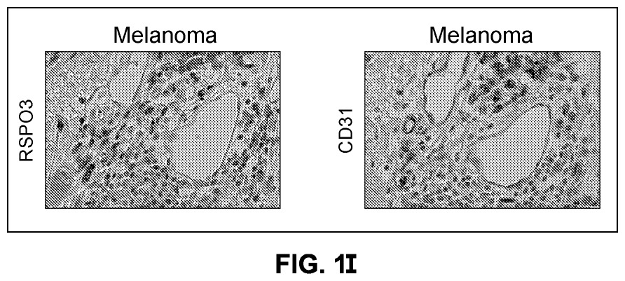

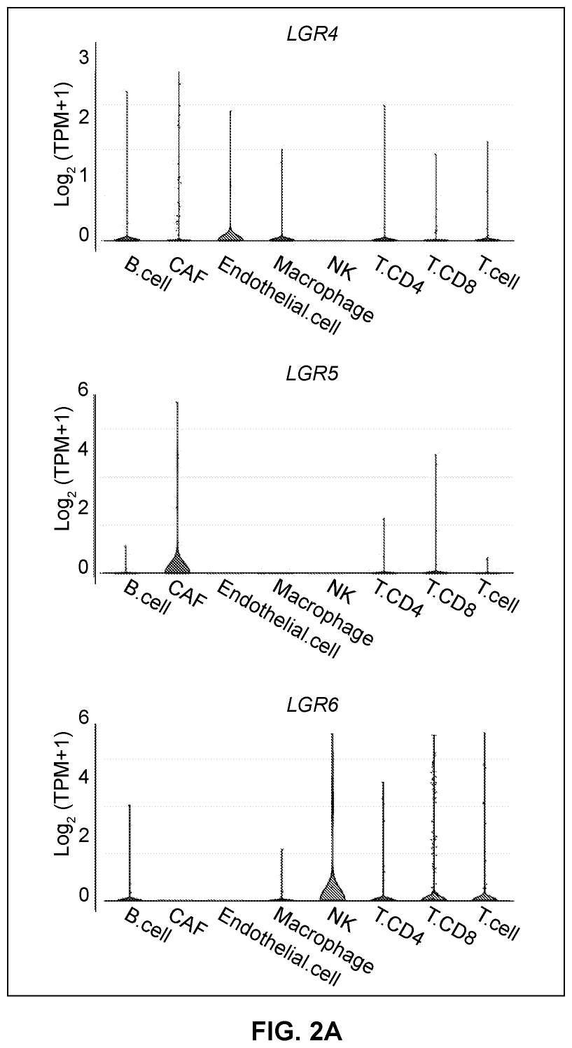

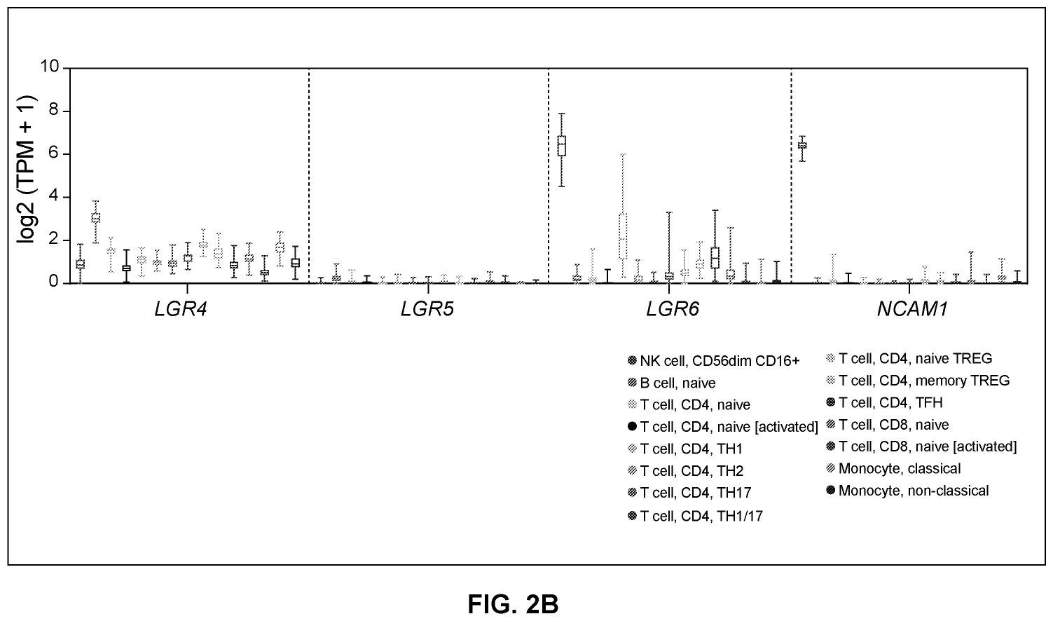

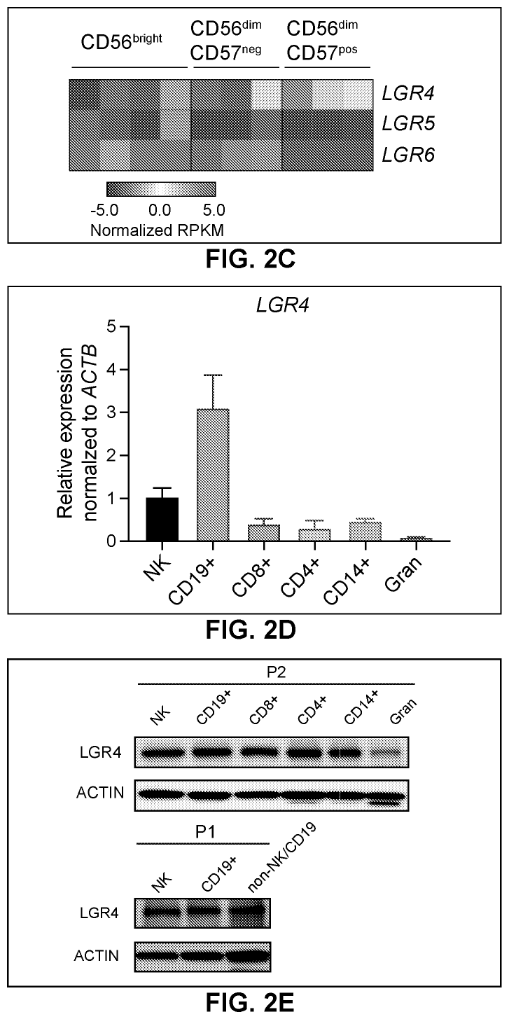

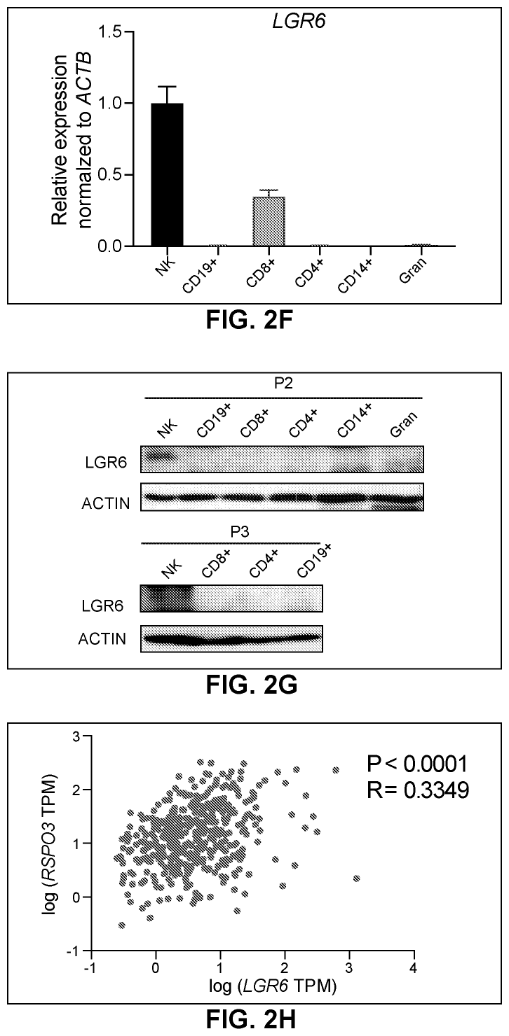

Applicant next investigated the expression of R-spondin receptors in the TME. LGR4, LGR5, and LGR6 are considered the obligate high-affinity receptors for R-spondins 1-4^{[13,17]} By analyzing the single-cell RNA-seq dataset of human melanoma^{[25]}, Applicant found that NK cells were the predominant cell population that expressed LGR6 in the TME, while ECs and CAFs express an appreciable amount of LGR4 and LGR5, respectively (FIG. 2, A). To further investigate the expression pattern of LGRs 4-6 in normal NK cells and other immune cells, Applicant analyzed the Database of Immune Cell Expression (DICE), which covers 15 immune cell subtypes that include NK cells, B cells, T cells, and monocytes from 91 healthy donors^{[27]}. Among these cell populations, NK cells showed the most pronounced expression of LGR6, whose transcript level (median Transcripts Per Million (TPM)=88.55, 95% CI: 76.52-102.5) was comparable to that of NCAM1 (median TPM=84.04, 95% CI: 81.15-86.93) which encodes for the canonical NK-cell marker CD56 (FIG. 2, B). Meanwhile, CD4⁺ Th1 cells displayed a varied but overall lower level of LGR6 compared to NK cells (FIG. 2, B). For the other two receptors, appreciable expression of LGR4 could be observed in B cells and LGR5 was not abundantly expressed by any of these immune cells (FIG. 2B). Another dataset from the Primary Cell Atlas in BioGPS Dataset Library[28, 29], a meta-analysis of microarray datasets of 745 human primary cell samples, also showed that NK cells and, to a lesser extent, CD8⁺ T cells are the two cell populations that had prominent expression levels of LGR6 (data not shown). The other two known R-spondin receptors—LGR4 and LGR5—are mainly expressed by embryonic stem cells and mesoderm-mesenchymal stem cell-derived cell lineages. Interestingly, analysis of a transcriptome dataset of human peripheral blood NK cell subsets^{[30]} suggested that the mature and cytotoxic subset of NK cells (CD56^{dim}CD57⁺) had higher levels of LGR6 compared to the less mature and less cytotoxic subsets (CD56^{bright}) (log 2FC=5.69, P<0.0001) (FIG. 2C). Together, these evidence consistently suggest that NK cells have a high transcriptional level of LGR6.

To validate the findings from these bioinfo-analyses, Applicant purified human NK cells, CD19+ B cells, CD8+ T cells, CD4+ T cells, CD14+ monocytes, and granulocytes from the peripheral blood of healthy donors and performed RT-qPCR or WB assays. Supporting the results shown in FIG. 2, B, RT-qPCR analysis revealed over 3-fold greater level of LGR4 in B cells compared to that in the rest of the peripheral blood cell populations (FIG. 2, D). Protein level of LGR4 showed no dramatic differences between the peripheral blood mononuclear cell fractions but showed a much lower level in the granulocytes (FIG. 2, E). While LGR6 mRNA was detected in NK cells and, to a less extent, by CD8+ T cells, the LGR6 protein was substantially expressed by NK cells but not by any other peripheral blood mononuclear cells with a decent amount (FIG. 2, F-G). These results suggest LGR6 is prominently expressed by human NK cells. Consistent with these data, a moderate positive correlation (R=0.33, P<0.05) was observed for LGR6 with RSPO3 in TCGA dataset of melanoma (FIG. 2, H), indicating active R-spondin ligands-LGR6 interactions possibly existed within the tumor tissue. Collectively, these data suggest that LGR6 is highly expressed by human NK cells and the R-spondin3/LGR6 axis may serve as a signaling axis in the TME to regulate NK-cell mediated anti-tumor immunity in human cancers.

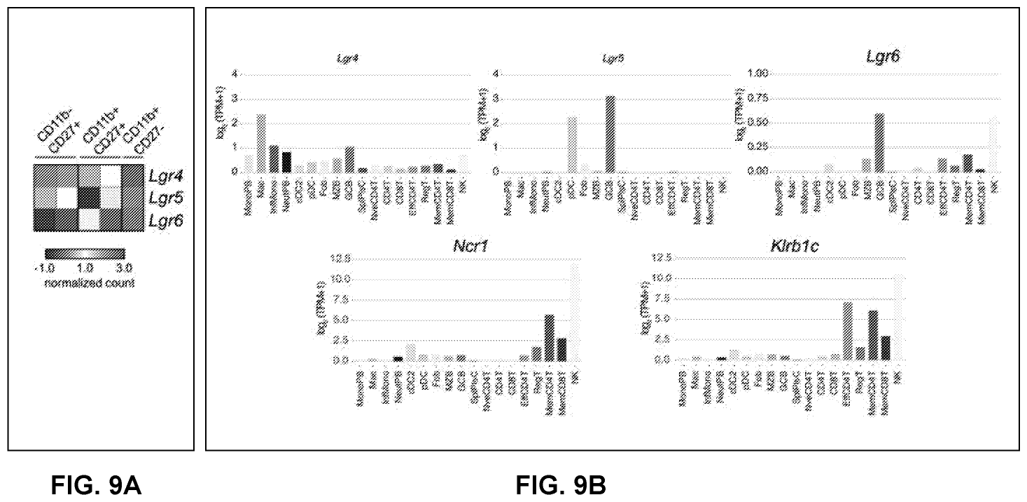

In mouse NK cells, as revealed by the immGen project dataset^{[31]}, a higher level of Lgr6, as well as Lgr4, was observed in the mature and cytotoxic subset (CD11b⁺CD27⁻) relative to the less mature and less cytotoxic subset (CD11b-CD27+) (Lgr6: log 2FC=4.09, P<0.05, Lgr4: log 2FC=3.03, P<0.05) (FIG. 9, A), suggesting a conserved regulatory mechanism for the expressions of R-spondin receptors that may exist between mouse and human NK cells. Notably, unlike in human samples, the overall transcription level of mouse Lgr6 in bulk NK cells, as revealed by the Haemopedia RNA-seq datasets^{[32]}, was not as abundant as that of mouse NK cell marker genes, such as Ncr1, Klrb1c (FIG. 9, B), implying differential functional significance for the LGR6-mediated signaling pathway in the associated biological processes between the two species.

Exogenous Expression of R-Spondin3 in the TME Inhibits Tumor Progression

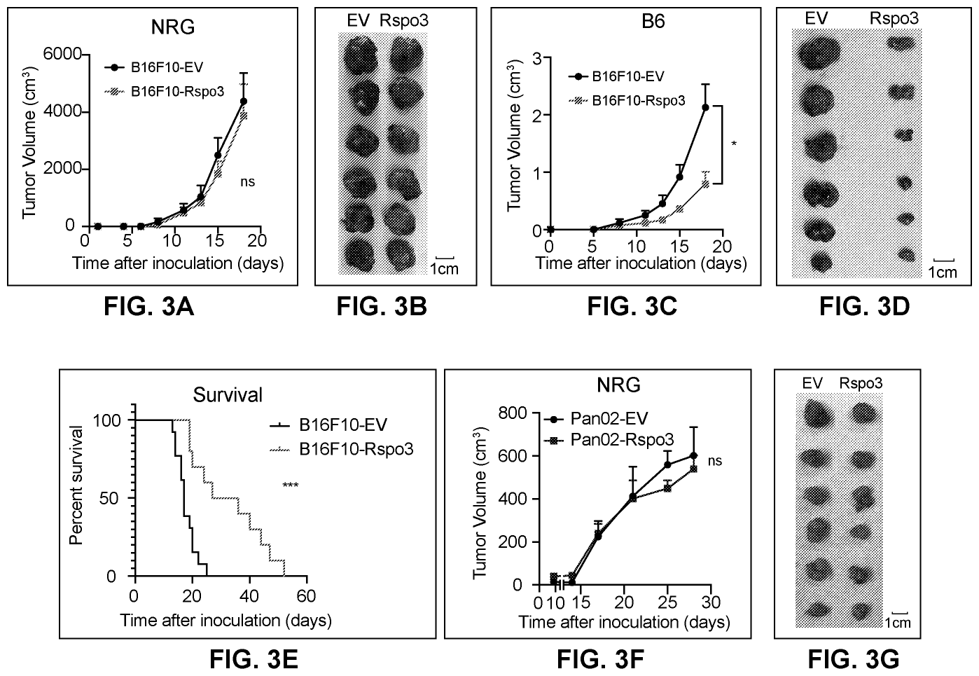

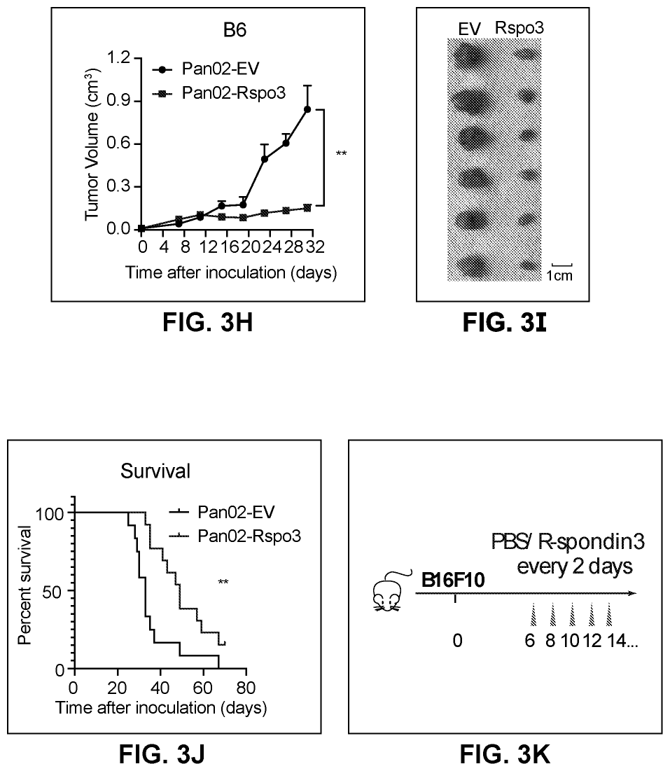

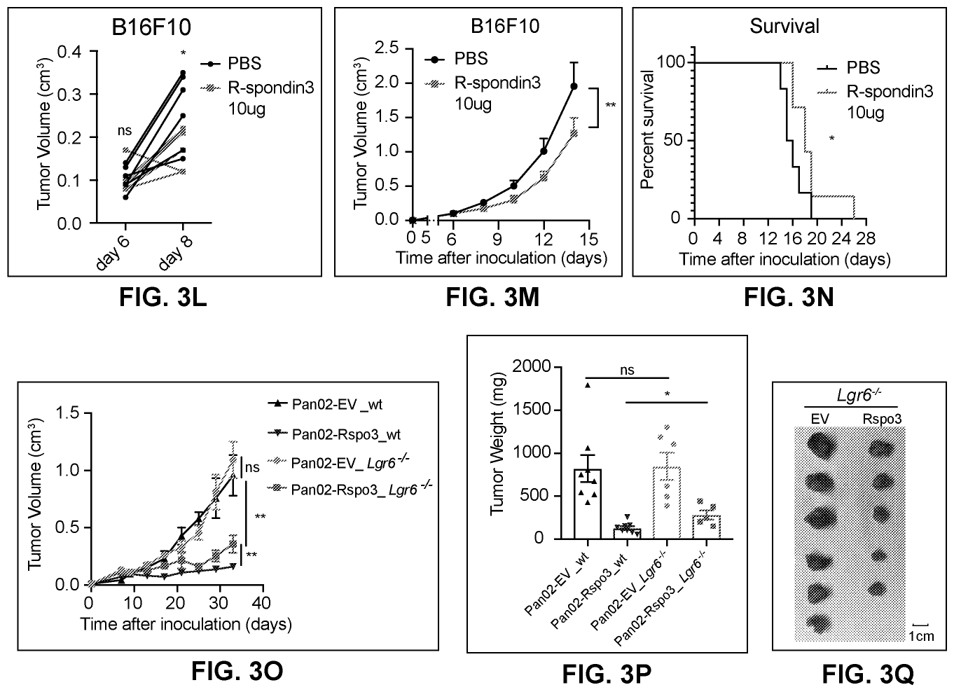

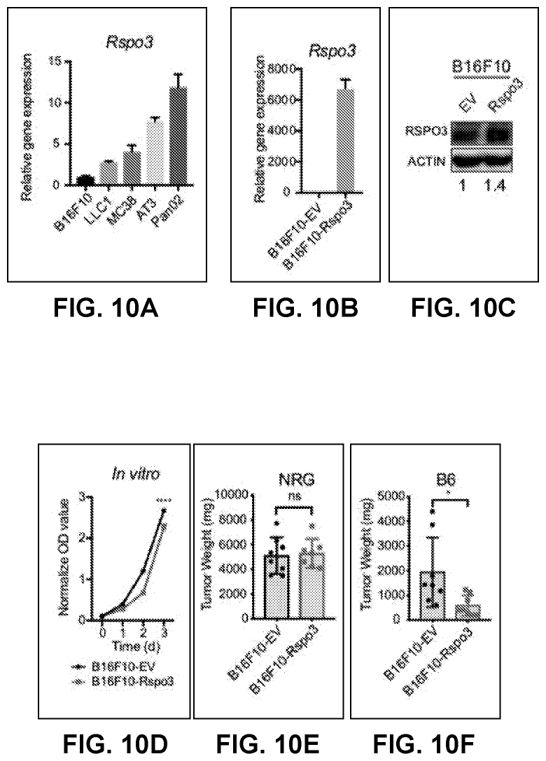

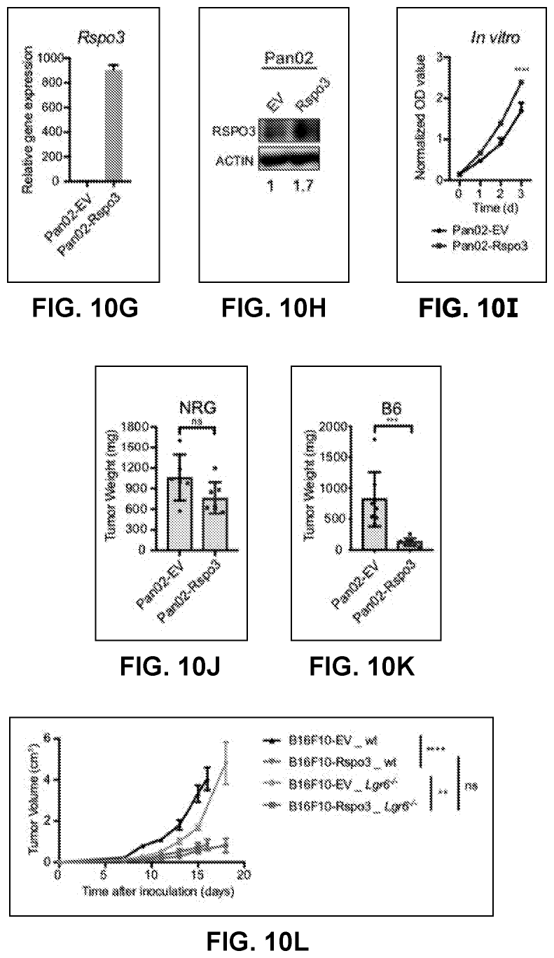

To investigate whether an increased level of R-spondin3 in the TME affects the tumor progression, Applicant generated mouse melanoma tumor cell line B16F10 overexpressing R-spondin3. The endogenous expression level of RSPO3 in B16F10 was low compared to several other tumor cell lines analyzed (FIG. 10, A). B16F10 cells were transduced with empty vector (B16F10-EV) or vector expressing R-spondin3 (B16F10-Rspo3) (FIG. 10, B-C). The two lines showed marginal growth difference in vitro (FIG. 10, D) and no growth difference in vivo in the immunodeficient NRG mice (NOD-Rag1^{null} IL2rg^{null}, NOD rag gamma) that lack both innate and adaptive immunity (FIG. 3A-B, FIG. 10, E). Notably, in the immune-competent syngeneic mice, B16F10-Rspo3 group showed substantially impaired tumor progression (FIG. 3C-D, FIG. 10, F) and prolonged overall survival (FIG. 3E) relative to the B16F10-EV group. Similar effects were recapitulated in a mouse pancreatic carcinoma cell line Pan02, in which the overexpression of R-spondin3 (FIG. 10, G-H) showed marginal effects on in vitro growth (FIG. 10, I) and in vivo growth in NRG mice (FIG. 3, F-G; FIG. 10, J), but inhibited the tumor progression in syngeneic B6 mice (FIG. 3H-J, FIG. 10, K). The tumors of Pan02-Rspo3 were also more movable and with clearer boundaries from surrounding tissues compared to Pan02-EV tumors, indicating less invasiveness. To further confirm the role of R-spondin3 in inhibiting tumor progression, Applicant performed intra-tumor injections of R-spondin3 protein to B16F10 syngeneic tumor models (FIG. 3, K). The treatment was effective in suppressing tumor growth and extending survival (FIG. 3, L-N). In the Lgr6^{-/-} mice, the exogenously expressed R-spondin3-mediated tumor suppression could still be observed in both tumor models, while the effect was diminished in the Pan02 model as shown by an enhanced tumor progression of Pan02-Rspo3 tumors in the Lgr6-1 mice relative to that in the wild type mice (FIG. 3 O-Q, FIG. 10. L), suggesting LGR6 partially mediated the tumor suppression caused by enhanced R-spondin3 levels in the TME in a tumor-specific fashion. Given a neglectable LGR6 protein expression in mouse NK cells (data not shown), the LGR6-mediated tumor suppression observed in the mouse tumor model may not necessarily be mediated through NK cells.

Exogenous expression of R-spondin3 in the TME enhances anti-tumor immunity.

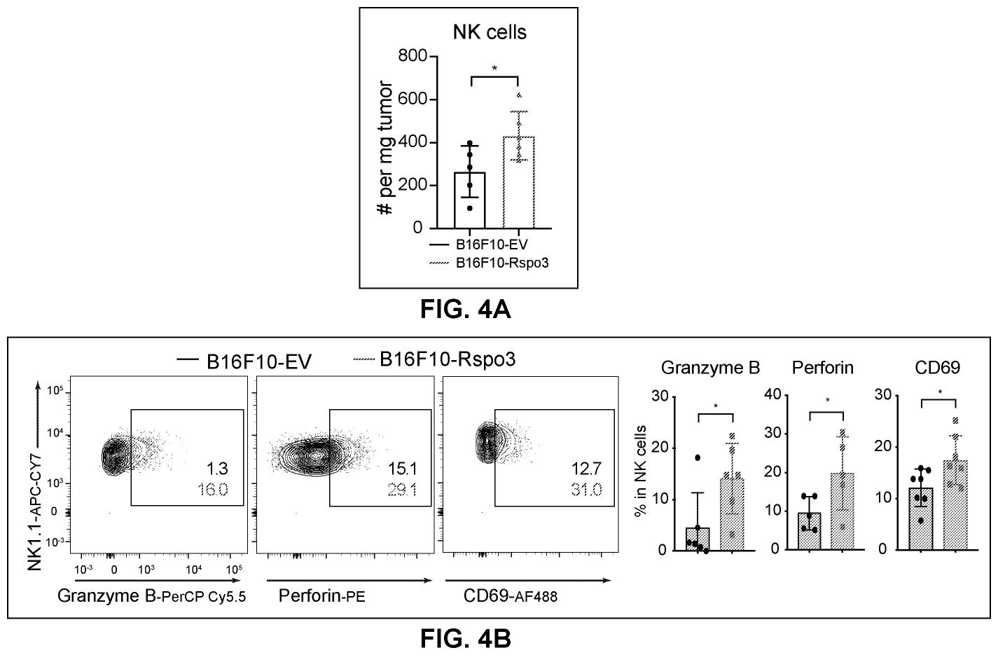

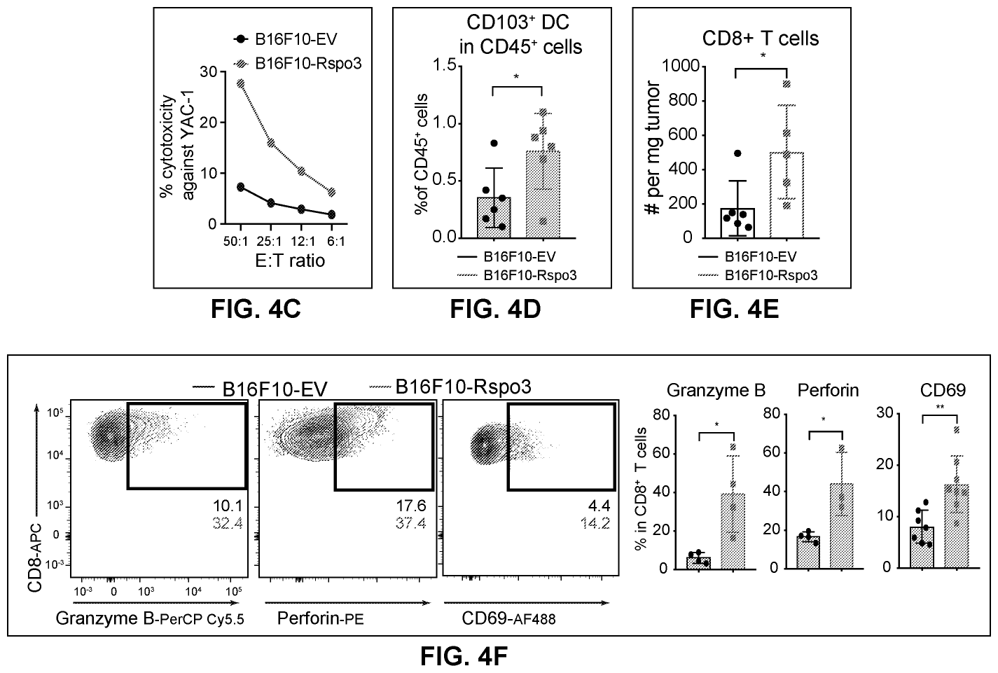

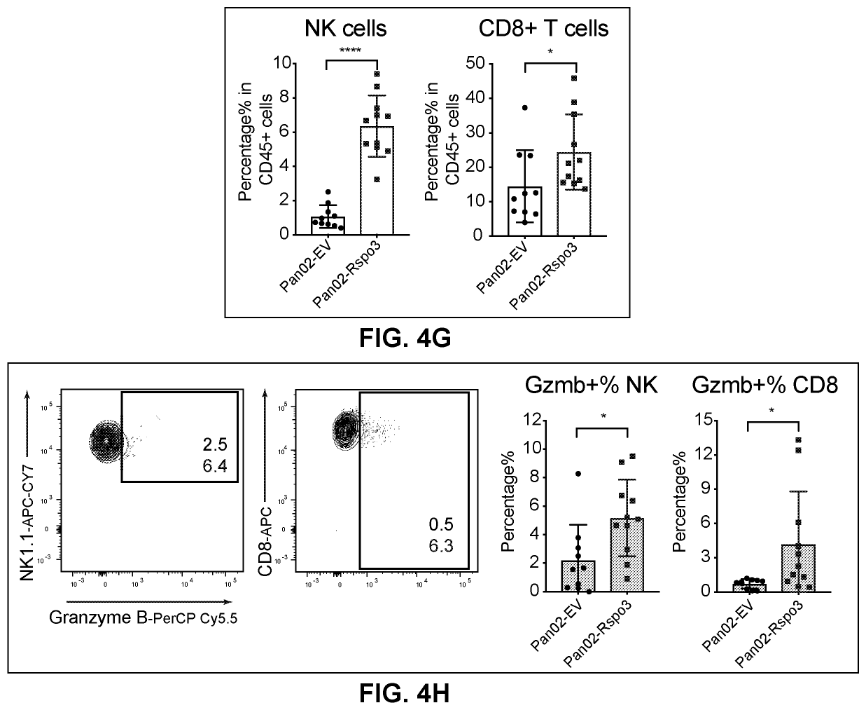

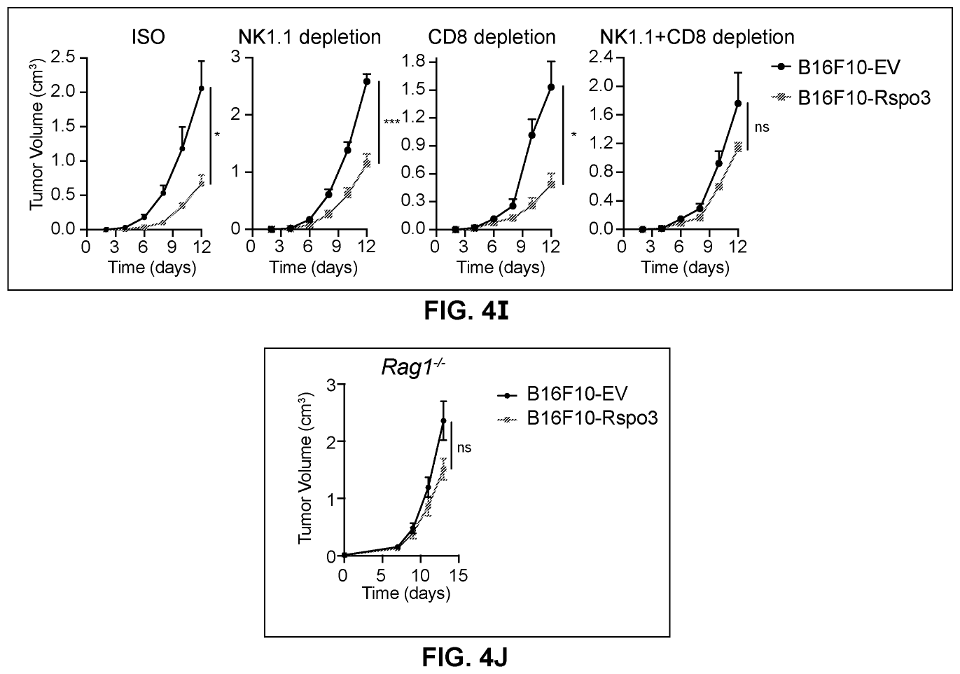

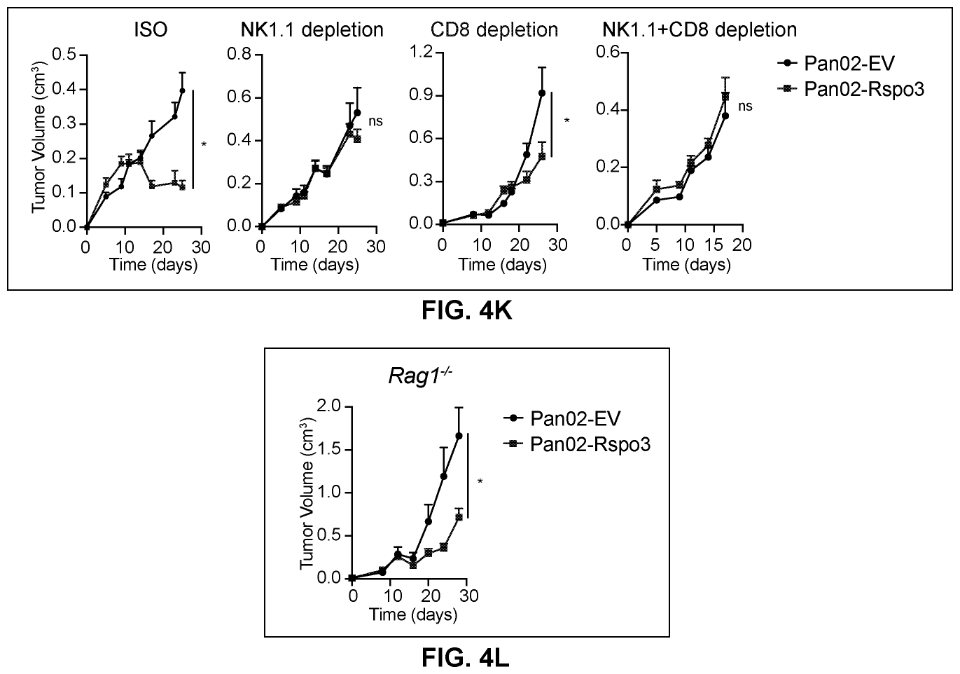

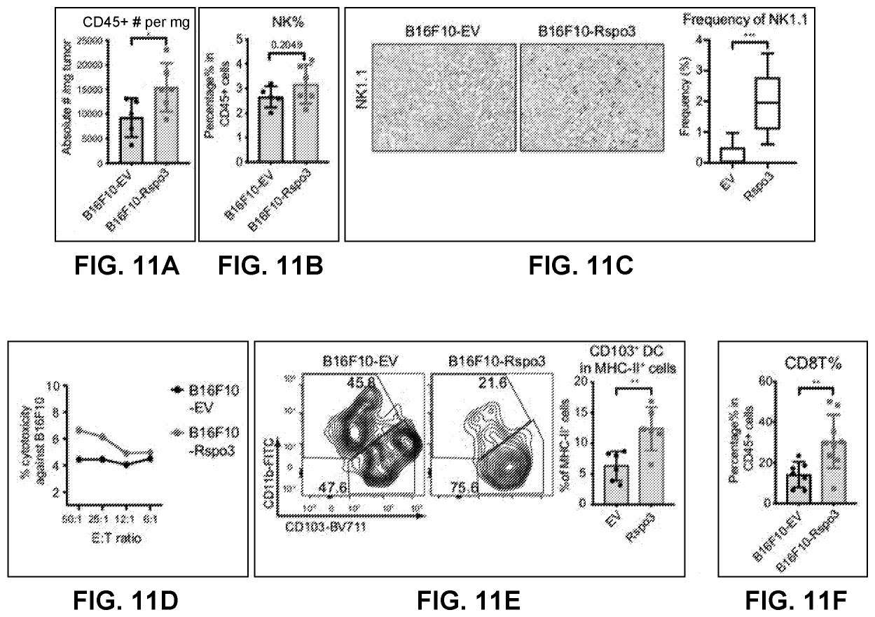

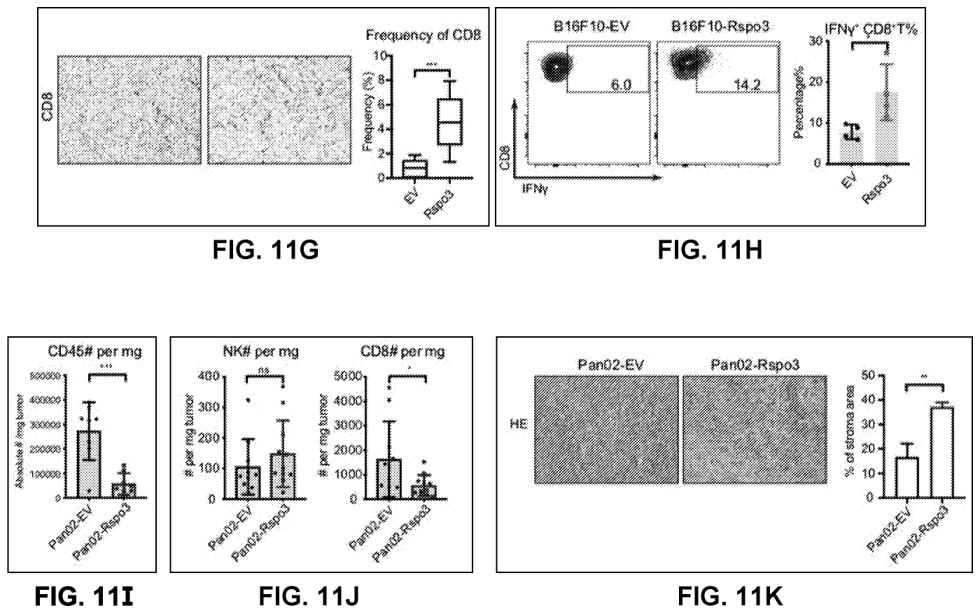

To investigate whether R-spondin3 in the TME affects the NK-cell and overall anti-tumor immunity, Applicant analyzed the tumor-infiltrating immune cells from both B16F10-EV and B16F10-Rspo3 tumors. An increased infiltration of CD45⁺ immune cells and NK cells was observed by both flow cytometry analysis and immunohistochemistry (FIG. 4A, FIG. 11, A-C). In addition, enhanced expressions of cytotoxic molecules granzyme B and perforin and activation marker CD69 were observed in the NK cells from B16F10-Rspo3 tumors (FIG. 4B). In vitro killing capacity against B16F10 cells or YAC-1 cells, an NK-cell sensitive MHC-I low-expressing lymphoma cell line, of the NK cells derived from B16F10-Rspo3 tumors were stronger compared to those derived from the B16F10-EV tumors (FIG. 4C, FIG. 11, D), indicating a better NK cell functionality. Furthermore, in the B16F10-Rspo3 tumors, Applicant observed an increased proportion of CD103⁺ cDC1s (FIG. 4, D; FIG. 11, E) and CD8⁺ T cells (FIG. 4, E; FIG. 11, F-G), whose expressions of granzyme B, perforin, CD69, and IFN-γ were also increased (FIG. 4, F, FIG. 11, H) compared to those in the B16F10-EV tumors, indicating a better overall anti-cancer immunity. Similarly, in the Pan02 pancreatic cancer model, increased percentages of NK cells and CD8⁺ T cells in the CD45⁺ immune cell population, with enhanced expressions of granzyme B, were detected in the Pan02-Rspo3 group relative to the Pan02-EV group (FIG. 4G-H), although enhanced infiltrations could not be observed when measured as absolute infiltrating cell number per tumor weight (FIG. 11, I-J), which could be related with an increased amount of stromal tissue observed in the Pan02-Rspo3 tumors (FIG. 11, K). Collectively, these data indicate that increasing R-spondin3 levels in the TME enhances the NK-cell anti-tumor immunity and promotes better overall anti-tumor responses.

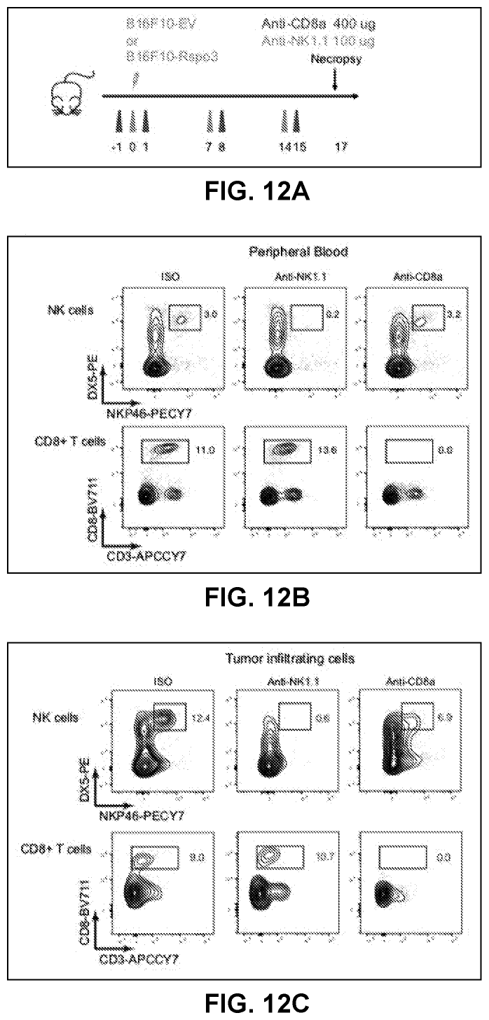

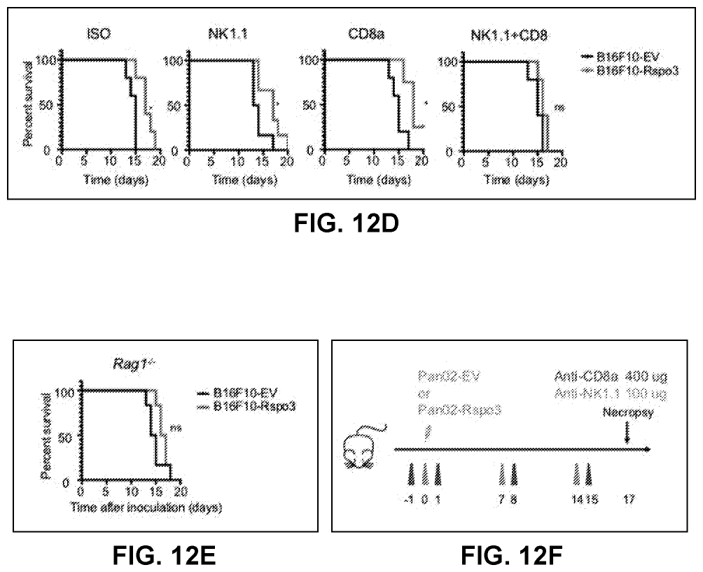

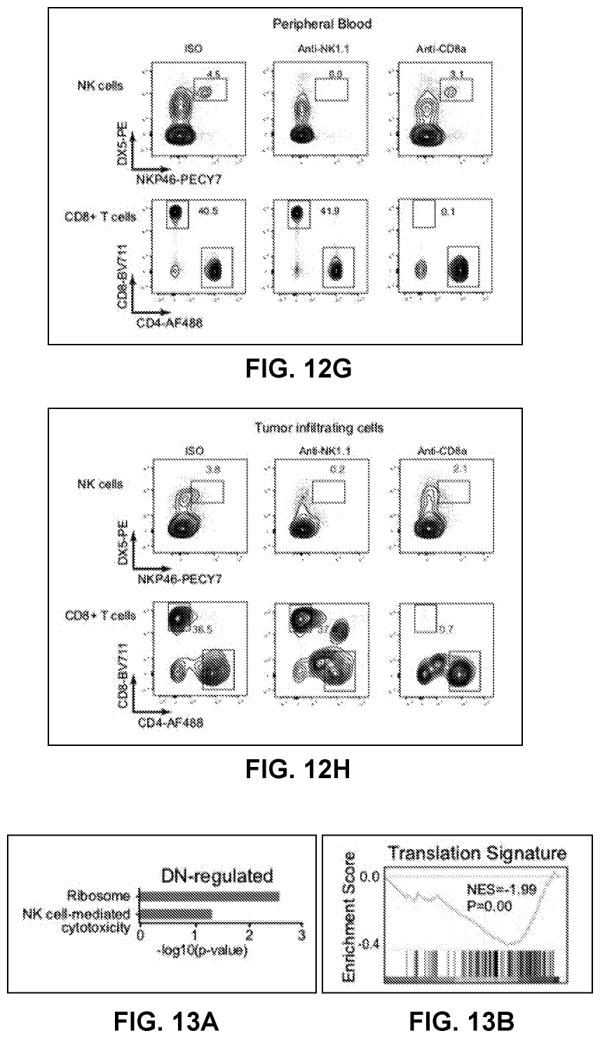

To dissect the roles of different immune cells in the TME in the R-spondin3-meditated tumor suppression, Applicant inoculated B16F10-EV and B16F10-Rspo3 tumors in mice lacking CD8⁺ T cells and/or NK cells by injecting the anti-CD8a and/or anti-NK1.1 depleting antibodies (FIG. 12, A-C). Depletion of both CD8⁺ T and NK cells, but not by depletion of either cell type alone, abrogated the tumor-suppressive effect of R-spondin3 exogenous expression (FIG. 4, I, FIG. 12, D), indicating that NK cells and CD8⁺ T cells are the major populations mediating tumor suppression and both can act at least partially independently on each other. Further functional dissection using Rag1^{-/-} mice, which lacks mature B cells and T cells, also abrogated tumor suppression (FIG. 4, J, FIG. 12, E), suggesting that other T cell subsets or B cells may also play roles here. Interestingly, in the Pan02 pancreatic cancer model, depleting NK cells alone was sufficient to abrogate the tumor suppression, and depleting CD8⁺ T cells alone or growth in Rag1^{-/-} mice could just modestly but noticeably reduced R-spondin3-mediated tumor suppression (FIG. 4, K-L, FIG. 12, F-H). These results suggested that both NK cells and CD8⁺ T cells contributed to the R-spondin3-mediated tumor suppression in this pancreatic carcinoma tumor model and there existed a T-cell-independent effect. Together, these data indicate that both NK cells and CD8⁺ T cells mediate the tumor-suppressive effects of R-spondin3.

R-spondin3 promotes MYC expression of NK cells in the TME.

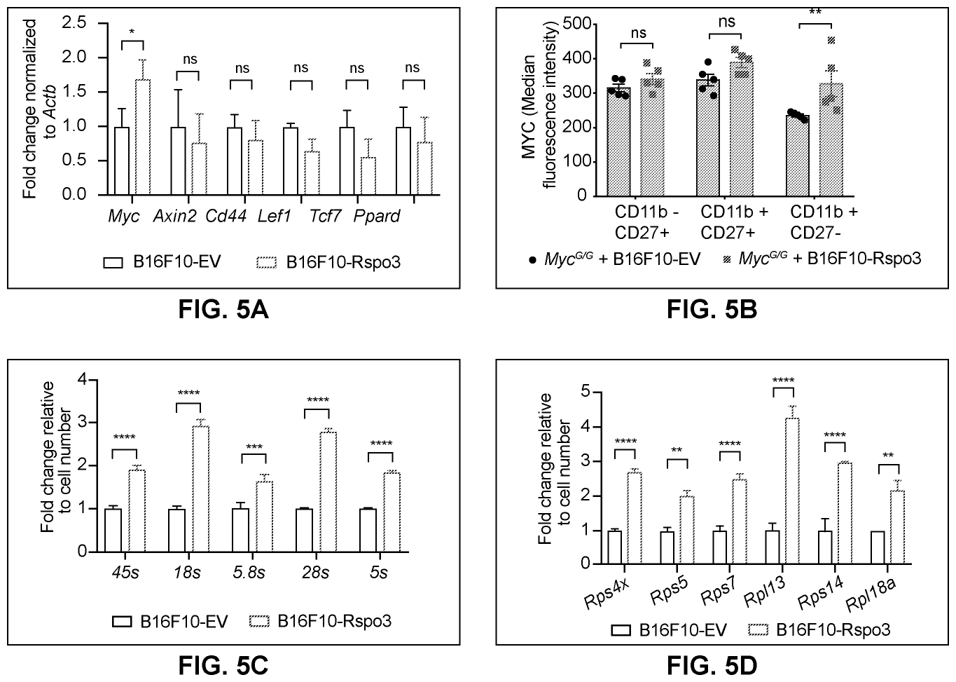

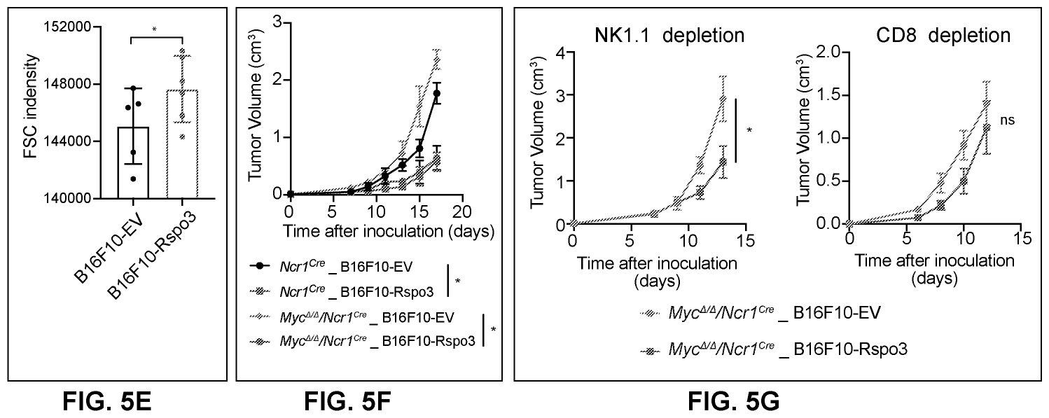

It is believed that R-spondins are able to potentiate the canonical Wnt signaling activity, which upregulates the expressions of a series of target genes in a cell-type- and context-specific manner^{[6,33]}. To explore the mechanism by which the R-spondin3 promote the anti-tumor immunity in the TME, Applicant sorted NK cells from the B16F10-EV and B16F10-Rspo3 tumors and measured the expression levels of several known Wnt target genes, including Myc, Axin2, Cd44, Lef1, Tcf7, Ppard, Mmp7, and Ccnd1. Among the detectable genes, Myc is significantly upregulated in the NK cells from the B16F10-Rspo3 tumor relative to the B16F10-EV tumor (FIG. 5A). Using syngeneic recipients of Myc^{G/G} mice, in which the endogenous Myc locus has been modified to encode a GFP-MYC fusion protein enabling the measurement of MYC expression with GFP signal intensities, Applicant further confirmed an increased MYC protein level in the tumor-infiltrating NK cells from the B16F10-Rspo3 tumors compared to B16F10-EV tumors (FIG. 5B). Thus, R-spondin3 promotes MYC expression in NK cells in the TME.

Applicant next explored the functional impact of altered MYC expression of NK cells in the R-spondin3-mediated tumor suppression. Implications from a Myc conditional knock-out mouse model—Myc^{Δ/Δ}/Ncr1^{Cre} mice—which depletes MYC expression in NK cells and restricted subsets of innate lymphoid cells^{[3,4]}, indicated that ribosome and NK cell-mediated cytotoxicity were the two most enriched pathways in the differentially down-regulated genes (FDR<0.05) by RNA-seq analysis of the isolated splenic NK cells from Myc^{Δ/Δ}/Ncr1^{Cre} mice (FIG. 13, A). Translation-associated gene set was also shown to be negatively enriched in the NK cells with Myc deletion (FIG. 13, B). These results suggested impaired ribosomal biogenesis as a feature for the NK cells with MYC deficiency. To determine whether the tumor-infiltrating NK cells from B16F10-Rspo3 tumors, which have enhanced MYC expression, also have enhanced ribosomal biogenesis compared to that from B16F10-EV tumors, quantitative RT-PCR was performed using the NK cells sorted from these tumors. Results showed increased expressions of rRNAs and mRNA level of ribosomal proteins by the NK cells from B16F10-Rspo3 tumors compared to that from the B16F10-EV tumors (FIG. 5, C-D). An increased FSC intensity revealed in flow cytometric analysis, a phenomenon usually observed in cells with enhanced ribosomal biogenesis associated with larger cell sizes, was also seen for the tumor-infiltrating NK cells in B16F10-Rspo3 tumors relative to −EV tumors (FIG. 5, E). Together, these indicate a stronger ribosomal biogenesis capacity of the NK cells in TME with a higher level of R-spondin3.

To determine whether MYC expression in NK cells is required for the R-spondin3-mediated tumor suppression, B16F10 tumors were inoculated to Myc^{Δ/Δ}/Ncr1^{Cre} mice and Ncr1^{Cre} controls. While the R-spondin3-mediated tumor suppression could still be observed in the Myc^{Δ/Δ}/Ncr1^{Cre} mice (FIG. 5F), which corresponded to the results shown in FIG. 4E that depleting NK cells alone was not enough to abrogate tumor suppression in wild type mice, Applicant found the tumor suppression was abrogated in the Myc^{Δ/Δ}/Ncr1^{Cre} mice with CD8⁺ T cell-depleted (FIG. 5G), suggesting MYC expression in NK cells is required for the contribution of NK cells to the R-spondin3-mediated effects.

R-Spondin3 Sensitizes Tumors to PD-1 Blocking Therapy

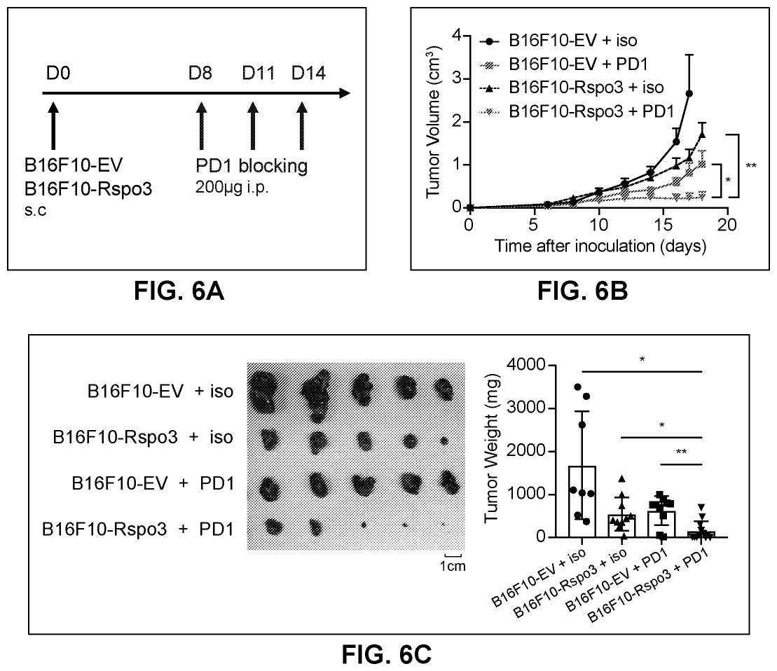

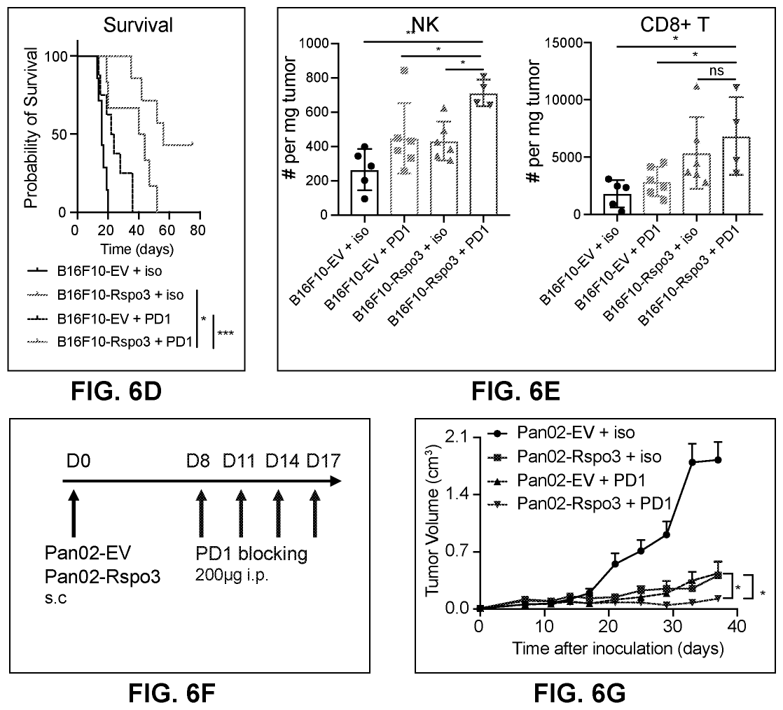

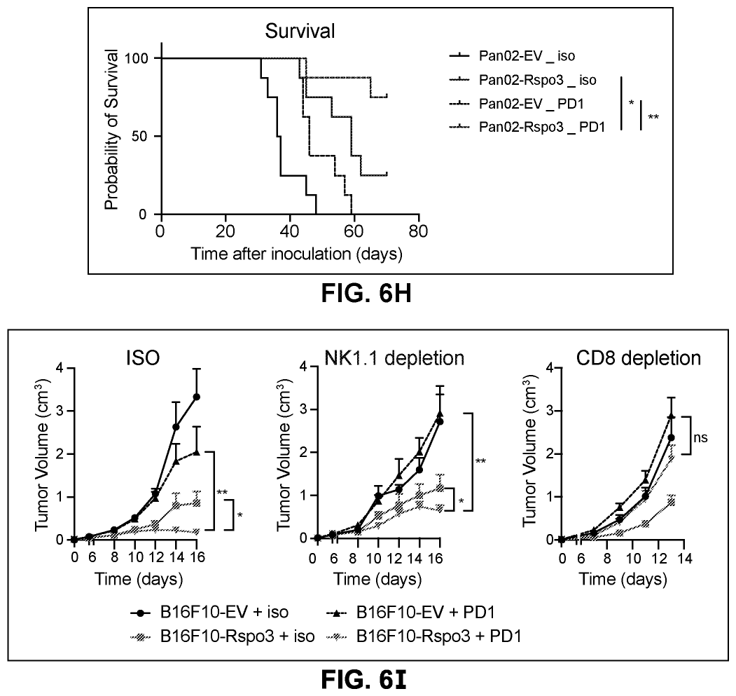

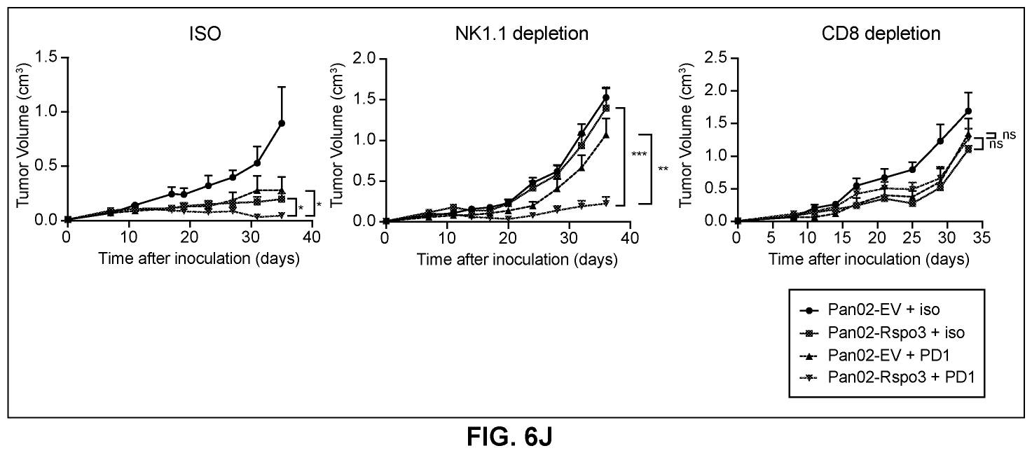

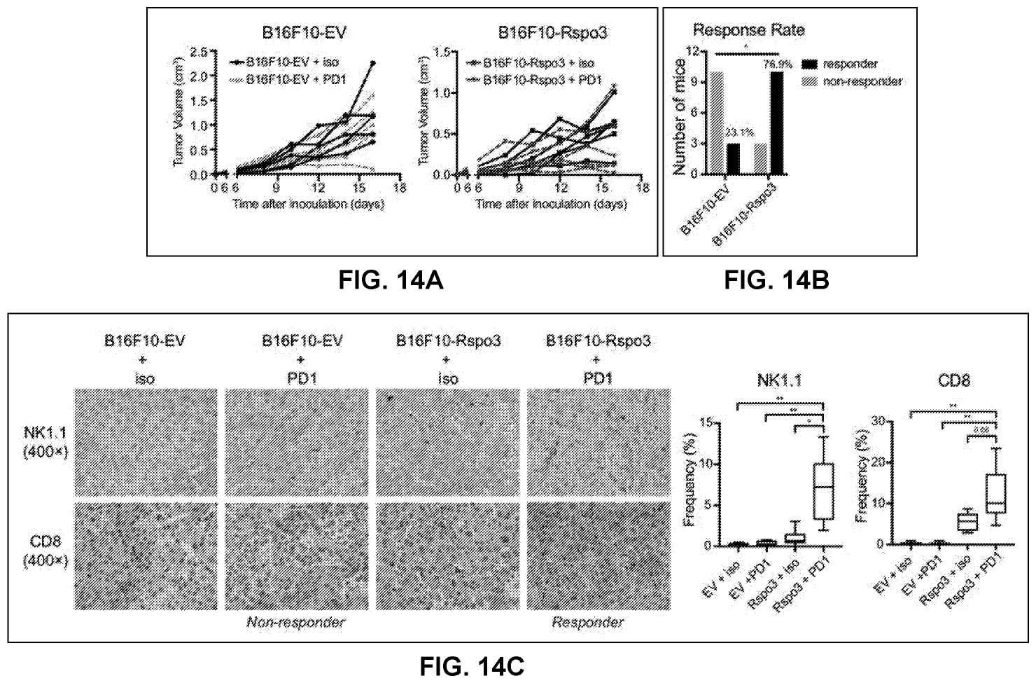

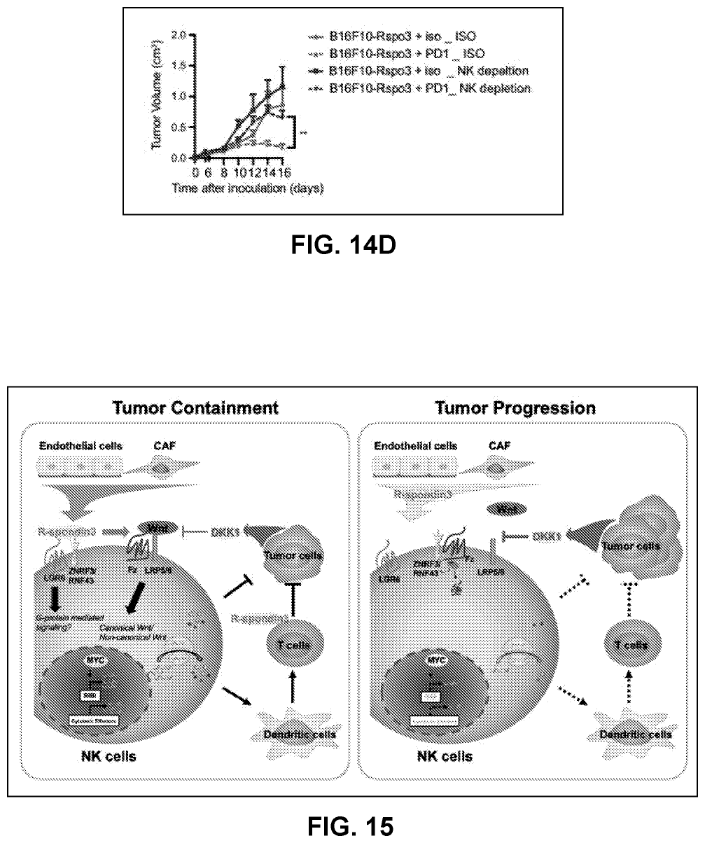

Given that NK cell activity and immune cell frequencies associate with patient responses to immune checkpoint inhibitors^{[2]}, Applicant next tested the therapeutic efficacy of anti-PD1 antibody in the R-spondin3-overexpressing B16F10 tumors, whose parental line is known to be resistant to anti-PD1 antibody therapy (FIG. 6, A). Results showed a sensitization of B16F10 tumors with R-spondin3 overexpression to anti-PD1 antibody treatment (FIG. 6, B), with some B16F10-Rspo3 tumors got nearly completed rejected (FIG. 6, C). The response rate of B16F10-Rspo3 tumors to anti-PD1 therapy was significantly higher compared to B16F10-EV tumors (FIG. 14, A-B). Survival of mice inoculated with B16F10-Rspo3 tumors with anti-PD1 therapy was substantially extended, with some achieved durable tumor remission (FIG. 6, D). The percentages of tumor-infiltrating NK cells and CD8⁺ T cells were both increased in the B16F10-Rspo3 tumors with anti-PD1 antibody treatment compared to other groups (FIG. 6, E, FIG. 14, C), indicating a better infiltration of cytotoxic cells. Further, therapeutic merits could also be observed in the Pan02-Rspo3 tumors with anti-PD1 antibody treatment (FIG. 6, F-H), indicating R-spondin3 and anti-PD1 therapy cooperatively enhance tumor control.

Applicant next dissected the contribution of NK cells and CD8⁺ T cells to the combinatory effect of R-spondin3 with anti-PD1 therapy. Results showed that depletion of CD8⁺ T cells completely abrogated the exceptional outcome achieved by anti-PD1 treatment in −Rspo3 tumors in both tumor models (FIG. 6, I-J), suggesting a dependence on CD8⁺ T cells for the combinatory effects of anti-PD1 therapy with increased tumor R-spondin3 level. Although this combinatory effect could still be observed after NK cell depletion (FIG. 6, I-J), the strength was much diminished in the B16F10 model (FIG. 14, D), suggesting NK cells also play a role here. Together, these data suggest a robust sensitization for PD-1 blocking therapy by enhanced R-spondin3 level in the TME.

Discussion

In this study, Applicant identified R-spondin3 and R-spondin1 derived from ECs/CAFs in the TME as regulators for anti-tumor immunity to affect cancer outcomes and sensitivity to immune checkpoint inhibitors. The expression of LGR6, a high-affinity receptor for R-spondins, is prominently expressed in human NK cells. Mechanistically, R-spondin3 enhances the MYC and ribosomal biogenesis gene expressions of NK cells in the tumor tissues.

Wnt signaling is delicately regulated by a variety of positive or negative regulators with temporospatial specificity. DKK1 is a secreted Wnt signaling negative regulator. Being a target gene of Wnt signaling, DKK1 is highly secreted by cancer cells with Wnt signaling aberrant activation^{[35]}. On the other hand, previous literature has shown cancers with aberrant β-catenin activation present immune deserts lacking infiltration of immune cells[11′ 12]. Interestingly, R-spondins synergize with Wnt proteins to activate canonical Wnt signaling with particular potency in the presence of DKK1^{[16]}. These collectively suggest that the levels of R-spondins are reasonable with the capacity to modulate the activity of canonical Wnt signaling in the TME. This could particularly be the case for non-cancer cells in the TME including anti-tumor immune cells, whose activation of Wnt signaling are not like in cancer cells that are determined by intrinsic mutations or aberrant activation, but are affected, to a greater extent, by the alterations of signals in the TME. These may also partially explain why numerous drugs inhibiting Wnt/MYC signaling showed good efficacy in vitro, while compromised in vivo^{[36]}.

The gene expressions of R-spondins are widely reduced across multiple cancers shown in TCGA data, while the underlying mechanism remains unclear. It is possible that R-spondin proteins are essential sustaining factors for NK cells in the TME and downregulating the expression levels of R-spondins may be a key mechanism for cancer cells to evade anti-tumor immunity. Applicant's data showed positive correlations of RSPO3 and, to a lesser extent, RSPO1 with immune cell signatures and cancer outcomes, while Applicant did not observe significant correlations for RSPO2 and RSPO4. This selectivity could be related to a more abundant expression of RSPO3 in tumor tissues compared to other RSPO genes (FIG. 1B). Although all four R-spondins are able to potentiate Wnt signaling, their expression patterns and phenotypes shown in knockout mice have striking differences^{[37]}, suggesting distinct roles for the four R-spondin members in modulating a variety of biological processes.

Applicant's data showed a pronounced expression of LGR6 in human NK cells, particularly by the more mature and cytotoxic subsets. The finding is of notable interest given a wide belief that the three B-type LGRs (LGR4/5/6) mainly regulate embryonic development and adult stem cell self-renewing as determined by their unique expression patterns majorly observed in stem/progenitor cell populations^{[16]}. Interestingly, in contrast to the neonatal lethality found in both Lgr4 null and Lgr5 null mutations mice, Lgr6 knockout mice are healthy and fertile^{[38]}, implying an essential difference for the biological functions of LGR6 from the other two LGRs. Future studies will be of great interests to clarify whether LGR6, like its expression in other tissues, marks specific NK cell populations with self-renewing capacities, such as memory NK cells; or whether it functions, like most other GPCRs that are highly specialized, to regulate certain NK-cell biological functions through G-proteins-mediated signaling, a general mechanism used by GPCRs while seems not yet identified to be used by the three B-type LGRs^{[13]}.

The R-spondins bind to LGR4/5/6 with high affinity through their furin-2 repeat, which allows the other furin repeat in R-spondin to interact with RNF43/ZNRF3, a membrane E3 ubiquitin ligase complex that removes Wnt receptors from cell surface. The subsequent endocytosis of the R-spondin-LGR-RNF43/ZNRF3 complex in turn leads to membrane clearance of the E3 ligases and persistence of Wnt receptors on the cell surface, thereby promoting Wnt signaling strength and duration^{[16,39]}. Of note, LGR-independent enhancement of Wnt signaling has also been reported recently for RSPO2 and RSPO3, which were determined by direct interaction of R-spondins with RNF43/ZNRF3^{[17,18]}. Interestingly, while RNF43/ZNRF3 homologs exist in invertebrates, the R-spondin/LGR/RNF43 module is considered as a relatively recent evolutionary “add-on” seen only in vertebrates and largely dedicated to adult stem cells^{[40]}. In this regard, although the pronounced LGR6 expression in human NK cells is highly likely to mediate profound biological functions in human cancers, the R-spondin3-mediated tumor suppression revealed by Applicant's study in mouse tumor models is not necessarily to be mediated through LGR6, as shown in the data of a minor or no rescue of R-spondin3-mediated tumor suppression in Lgr6^{-/-} mice (FIG. 3, O-Q, FIG. 10, L). In contrast, LGR-independent signaling or LGR4/5 mediated signaling in other cell components in the TME may play roles here. Applicant's observation of a neglectable LGR6 protein expression in mouse NK cells along with a peak expression bar shown in the NK cells relative to other cell types by RNA-seq analysis (FIG. 9, C) may provide one evidence for the occurrence of the evolution of this LGR-mediated exquisite modulation of Wnt signaling in an early phase shown in mice.

MYC, as the key target gene of canonical Wnt signaling pathway, is a master regulator controlling a variety of cellular processes, which includes ribosomal biogenesis that regulates mRNA translation. The expression of MYC has been shown essential for NK cell metabolism and functional status^{[41,42]}. Reduced MYC expression in the peripheral blood NK cells of patients with cancers was also reported^{[43]}. Of note, one hallmark of NK cells is that they maintain abundant mRNA levels of cytotoxic molecules at rest, and a ready-to-go ribosomal biogenesis machinery that ensures prompt translation of cytotoxic molecules when encountering target cells is critically needed for their innate killing capacity^{[44]}. Thus, Applicant's data that showed enhanced MYC and ribosomal biogenesis gene expressions in the NK cells with increased R-spondin3 in TME provides mechanistic insights on how R-spondin3 promotes anti-tumor immunity. As the exact molecular basis of R-spondin-mediated signaling modulation in this context was unspecified, further studies may be performed to interrogate whether the enhanced MYC expression is a direct target of enhanced canonical Wnt signaling potentiated by R-spondin or a secondary consequence of an improved TME by R-spondin through activating other signaling pathways, such as non-canonical signaling. Given the use of a constitutive Ncr1^{Cre} in Applicant's study that could result in an altered NK cell compartment and function, potential confounding factors could be involved to affect the observed phenotypes. Thus, an inducible Ncr1-iCreER allele that has been reported recently^{[45]} could be used to study the NK cell biology in cancers.

Inhibition of the PD-1/PD-L1 pathway has become a very powerful therapeutic strategy that remarkably improved the prognosis of patients with cancers. However, resistance remains a hurdle for a broader application, with multiple mechanisms proposed to contribute, which include an inadequate T or NK cell infiltration. Regarding this, in our tumor models, enhanced R-spondin3 in the TME promoted a better infiltration of both T cells and NK cells in tumor tissues, which is likely to be one mechanism for the sensitization to anti-PD1 therapy. This combinatory effect is not only dependent on CD8⁺ T cells, depletion of NK cells also reduced the sensitivity to anti-PD1 antibody observed in the B16F10-Rspo3 tumor model, which could be due to a diminished tumor suppression by R-spondin3 and/or a consequence of immune checkpoint inhibitors to directly target NK cells^{[3,4]}. Further, depletion of CD8⁺ T cells completely eliminated any anti-tumor benefits observed in the B16F10-Rspo3 tumors, indicating complexity of the CD8⁺ T cell-independent mechanism by anti-PD1 antibody combined with R-spondin3. Future studies to investigate whether there is an altered expression of PD1 or PD-L1 on NK cells and CD8⁺ T cells will be of interest and informative to clarify underlying mechanisms.

Applicant's study provides supports for a translational potential of R-spondin proteins as immunotherapeutic agents to treat cancers. While the safety of R-spondins being therapeutic agents should take into consideration of their roles in regulating the differentiation and proliferation of adult stem cells and tumorigenesis^{[46,47]} Gene fusions involving RSPO3 or RSPO2 were previously identified in colon cancers, and anti-RSPO3 treatment was demonstrated to inhibit tumor growth in PTPRK-RSPO3-fusion-positive human tumor xenografts through mechanisms including regulating intestinal stem-cell function and promoting differentiation^{[48]}. Of note, although transgenic overexpression of RSPO3 or RSPO3 fusion genes could result in adenomatous growth of the intestine, these alone were not sufficient to promote continued tumor growth^{[49,50]}, supporting the observation that RSPO fusion genes always co-occur with either BRAF or KRAS mutation in colon cancers^{[47]}. Therefore, the strategy of utilizing R-spondins as immunotherapeutic agents remains promising.

To conclude, Applicant has identified a novel role of R-spondins in promoting anti-tumor immunity in the TME (FIG. 15).

### Methods

Mice. All mice were bred and housed in specific pathogen-free conditions in the animal barrier facility at the Cincinnati Children's Hospital Medical Center (CCHMC). All animal studies were conducted in accordance with an approved Institutional Animal Care and Use Committee protocol and federal regulations. Lgr6^{-/-} mice (Jackson stock #016934), NRG mice (NOD-Rag1^{null} IL2rg^{null}, NOD rag gamma), Rag1^{-/-} mice (Jackson stock #002216), C57BL/6 mice (Jackson stock #000664), C57BL/6 congenic BoyJ mice were purchased from Jackson or Comprehensive Mouse and Cancer Core of CCHMC. All BoyJ mice used are confirmed with the expression of NKp46 by flow cytometry analysis with peripheral blood samples. The Myc^{G/G} mice were a kind gift from Dr. H. Leighton Grimes at Cincinnati Children's Hospital Medical Center, OH, USA. Myc^{f/f} mice and Ncr1^{Cre} mice were backcrossed to C57BL/6 background at Applicant's lab. All mice used were 8 to 12 weeks old. Age and sex matching were performed for each independent experiment. The Myc^{G/G} mice, Myc^{Δ/Δ}/Ncr1^{Cre} mice were born at the expected Mendelian ratios and showed normal WBC, hemoglobin, and platelet counts.

Transient Transfection and Retrovirus Infection. ORF clone of mouse Rspo3 (NM_028351.3) in the pcDNA3.1 vector was purchased from GenScript (Piscataway, NJ, USA). The full length of the ORF region was amplified with PCR using the primers: 5′-CTTGTCGACGCCACCATGCACTTGCGACTG-3′ (forward), (SEQ ID NO: 1) and 5′-GTCGAGAATTCTTATCACTTATCGTCGTCATC-3′ (reverse) (SEQ ID NO: 2) and cloned into the pMSCV-hpGK-GFP vector using the SalI and EcoRI restriction enzyme. Retroviruses were generated by calcium phosphate transient co-transfection of the retroviral vectors (MSCV-Rspo3-hpGK-eGFP or MSCV-hpGK-eGFP) with the packaging plasmids Gag and Eco-env into 293T cells. The supernatant was harvested at 48 hours and 72 hours and filtrated with a 0.45 um filter. B16F10 or Pan02 cells were plated in a 6-well plate one day before the transduction. On the day of transduction, 1 ml of the original media was kept and 2 ml of the retroviral supernatant was added. Polybrene was used at the final concentration of 6 ug/ml. Cells were centrifuged at 800 g for 90 minutes at RT. The GFP positive cells were sorted using flow cytometry two weeks after the transduction for further use.

Syngeneic Mouse Tumor Models. 7- to 12-week-old mice were used to establish syngeneic mouse tumor models. Mice were subcutaneously (s.c.) inoculated with B16F10 or Pan02 lines (5×10⁵ cells/mouse) into the right flank of the mouse. A caliper is used to measure the length and width of the tumor, and tumor volumes are estimated using the formula: [(length)×(width)×(width)]×0.52. The tumor volumes were monitored. Mice were killed before the tumor reached the maximum permitted size. Anti-PD1 antibody (29F.1A12) and isotype (2A3) were purchased from Bio X Cell, West Lebanon, NH, and administered 200 μg/mouse intraperitoneally at the indicated time point as described. Recombinant carrier-free mouse R-spondin3 protein (R&D, 4120-RS-025/CF) was used for intra-tumor injection with the regimen indicated. For immune cell depletion studies, antibodies against CD8a (YTS 169.4, Bio X cell), NK1.1 (PK136, Bio X Cell) were used. CD8a depletion (400 ug) was administered by intraperitoneal injection started on day −1, day 1, and was continued weekly for the duration of the experiment. NK1.1 depletion (100 ug) was administered by intraperitoneal injection started on day 0 and was continued weekly for the duration of the experiment. Lymphocyte depletions were confirmed in peripheral blood lymphocytes and tumor-infiltrating lymphocytes by flow cytometry with the following antibodies: CD8a (53-6.7) and NKp46 (29A1.4).

Cell Lines. B16F10 cell line was purchased from American Type Culture Collection (ATCC). Pan02 cell line was purchased from the Division of Cancer Treatment and Diagnosis (DCTD), National Cancer Institute. Both cell lines were actively cultured for less than four months after purchase and not further authenticated. Mycoplasma testing was performed at least every two months by Universal Mycoplasma Detection Kit (ATCC, 30-1012K), with the latest testing date on Jan. 5, 2021. The B16F10 cell line was cultured in DMEM (Thermo Fisher, 12430054) including 10% fetal bovine serum (Thermo Fisher, 16140-071) and 1× penicillin and streptomycin (Thermo Fisher, 15140-122). The Pan02 cell line was cultured in RPMI-1640 (Thermo Fisher, 21870-076) including 10% fetal bovine serum (Thermo Fisher, 16140-071) and 1× penicillin and streptomycin (Thermo Fisher, 15140-122). All cells were cultured at 37° C., 5% CO2.

Bio-info Analysis of Patient Transcriptome Data. Kendall's correlation matrix analysis was performed based on a total of 60 components of Wnt signaling pathway (Table 1) and NK cell signature of SKCM, PAAD, LUSC, HNSC, and visualized as heatmap after hierarchical clustering in Phantasus v1.5.1^{[1]}. For the correlation analyses between genes or gene signatures, Spearman's correlation analyses were performed in GEPIA2 using TCGA datasets^{[2]} and summarized in Prism 8.0.1. Survival analyses were performed with TCGA datasets in GEPIA2. The gene expressions in the single-cell RNA-seq datasets of melanoma or pancreatic carcinoma patients were visualized with R or in Broad Institute's Single Cell Portal (http://singlecell.broadinstitute.org/single_cell) using the previously reported datasets^{[3]}. DICE dataset was downloaded from https://dice-database.org. Data presented with Primary Cell Atlas in BioGPS Dataset was obtained from BioGPS portal (http://biogps.org/#goto=welcome). Statistical significance of gene expressions between NK cell subsets was performed with EdgeR algorithm by galaxy tool^{[4]}. Heatmaps were visualized with Phantasus^{[5]}.

TABLE 1

| TCGA Abbreviation | Abbreviation Full Name |
|---|---|
| ACC | Adrenocortical carcinoma |
| BLCA | Bladder Urothelial Carcinoma |
| BRCA | Breast invasive carcinoma |
| CESC | Cervical squamous cell carcinoma and |
| | endocervical adenocarcinoma |
| CHOL | Cholangio carcinoma |
| COAD | Colon adenocarcinoma |
| DLBC | Lymphoid Neoplasm Diffuse Large B-cell |
| | Lymphoma |
| ESCA | Esophageal carcinoma |
| GBM | Glioblastoma multiforme |
| HNSC | Head and Neck squamous cell carcinoma |
| KICH | Kidney Chromophobe |
| KIRC | Kidney renal clear cell carcinoma |
| KIRP | Kidney renal papillary cell carcinoma |
| LAML | Acute Myeloid Leukemia |
| LGG | Brain Lower Grade Glioma |
| LIHC | Liver hepatocellular carcinoma |
| LUAD | Lung adenocarcinoma |
| LUSC | Lung squamous cell carcinoma |
| MESO | Mesothelioma |
| OV | Ovarian serous cystadenocarcinoma |
| PAAD | Pancreatic adenocarcinoma |
| PCPG | Pheochromocytoma and Paraganglioma |
| PRAD | Prostate adenocarcinoma |
| READ | Rectum adenocarcinoma |
| SARC | Sarcoma |
| SKCM | Skin Cutaneous Melanoma |
| STAD | Stomach adenocarcinoma |
| TGCT | Testicular Germ Cell Tumors |
| THCA | Thyroid carcinoma |
| THYM | Thymoma |
| UCEC | Uterine Corpus Endometrial Carcinoma |
| UCS | Uterine Carcinosarcoma |
| UVM | Uveal Melanoma |

Wnt signaling pathway components

| | |
|---|---|
| WNT1 | FZD4 |
| WNT10A | FZD5 |
| WNT10B | FZD6 |
| WNT11 | FZD7 |
| WNT16 | FZD8 |
| WNT2 | FZD9 |
| WNT2B | FZD10 |
| WNT3 | RSPO1 |
| WNT3A | RSPO2 |
| WNT4 | RSPO3 |
| WNT5A | RSPO4 |
| WNT5B | DKK1 |
| WNT6 | DKK2 |
| WNT7A | DKK3 |
| WNT7B | DKK4 |
| WNT8A | SFRP1 |
| WNT8B | SFRP2 |
| WNT9A | SFRP4 |
| WNT9B | SFRP5 |
| FZD1 | LRP4 |
| FZD2 | LRP5 |
| FZD3 | LRP6 |

Flow Cytometry and Cell Sorting. Flow cytometry analysis and cell sorting were performed with FACS Canto, LSR Fortessa, or FACSAria instruments (BD Biosciences). Single-cell suspensions of mouse peripheral blood, bone marrow, spleen, and lymph node were obtained by forcing of organs through 70 μm cell strainer. Single-cell suspensions of tumors were digested in HBSS buffer in the presence of Collagenase D (Sigma, 2 mg/ml), Hyaluronidase (Sigma, 0.75 mg/ml), and DNaseI (Sigma, 0.4 mg/ml) for 45 min at 37° C. before passing through the cell strainer. Erythrocytes were then eliminated by RBC lysis buffer. Single-cell suspensions were used for surface staining in phosphate-buffered saline PBS containing 2% fetal bovine serum (FBS) and followed by intracellular staining or secondary staining if necessary. Fixation/Permeabilization Solution Kit (BD Biosciences) was used for intracellular staining of perforin, granzyme B, and IFN-γ. Antibodies were purchased from Biolegend, BD Bioscience, eBioscience, or Thermo Fisher: CD3 (145-2C11 or 17A2), NK1.1 (PK136), CD49b (DX5), CD11b (M1/70), CD27 (LG.3A10), NKp46 (29A1.4), CD107a (1D4B), IFN-γ (XMG1.2), Ly6G (1A8), B220 (RA3-6B2), CD8 (53-6.7), CD4 (GK1.5), CD115 (AFS98), CD25 (PC61), CD11c (HL3), MHC-II (M5/114.15.2), CD19 (6D5), mouse CD45 (30-F11), Ly6C (HK1.4), CD24 (30-F1), F4/80 (BM8), CD103 (2E7), CD69 (H1.2F3), MYC (Y69), perforin (eBioOMAK-D), granzyme B (QA16A02), BV421 goat anti-rabbit IgG, Alexa Fluor 488 donkey anti-rabbit IgG (H+L), streptavidin. 7-AAD (BD Biosciences, 559925) or Zombie Aqua Fixable Viability Kit (Biolegend, 423101) was used to exclude dead cells during analysis. For measurement of absolute cell number of tumor-infiltrating immune cells per tumor weight, CountBright Absolute Counting Beads (Thermo Fisher, C36950) were used. Data were analyzed using FlowJo software. All flow cytometric data were acquired using equipments maintained by the Research Flow Cytometry Core in the Division of Rheumatology at Cincinnati Children's Hospital Medical Center.

RNA Preparation and Real-Time qPCR. Bone marrow or spleen single-cell suspensions were prepared and stained as stated above before sorted into different populations using a FACSAria Cell Sorter (BD Biosciences). The purity of sorted cell populations was >95%. Sorted cells were lysed directly in RLT buffer from the RNeasy Micro kit (QIAGEN), and total RNA was extracted according to the manufacturer's instructions. Amounts of total RNA were measured using NanoDrop according to the manufacturer's instructions. cDNA was synthesized using the SuperScript III First-Strand Synthesis System for the RT-PCR Kit (Invitrogen). The cDNA was amplified using SYBR Green Master Mix (Life Technologies) with an Applied Biosystems Step One Plus thermal cycler (Applied Biosystems). Expression of target genes were determined using Atcb as internal control unless otherwise noted.

RNA-seq and Data Analysis. CD3⁻NK1.1⁺DX5⁺NK cells were sorted by flow cytometry from the splenic cells of three Myc^{f/f} and two Myc^{Δ/Δ}/Ncr1^{Cre} mice using FACSAria Cell Sorter (BD Biosciences). Total RNA was prepared as described above and submitted for RNA-seq analysis. Directional RNA-seq was performed by the Genomics, Epigenomics and Sequencing Core (GESC) at the University of Cincinnati. The RNA quality was determined by Bioanalyzer (Agilent, Santa Clara, CA). NEBNext Poly(A) mRNA Magnetic Isolation Module (New England BioLabs, Ipswich, MA) was used to isolate the polyA RNA. A total of 1 μg of good quality total RNA was used as input. The dUTP-based stranded library was prepared using the NEBNext Ultra II Directional RNA Library Prep Kit (New England BioLabs). The library was indexed and amplified under 8 PCR cycles. After library Bioanalyzer QC analysis and quantification, individually indexed and compatible libraries were proportionally pooled and sequenced using the Hiseq 1000 (Illumina, San Diego, CA). About 25 million pass filter reads per sample were generated under the sequencing setting of single read 1×51 bp.

Sequence reads were aligned to the reference genome using the TopHat aligner^{[6]} and aligned reads to each known transcript were counted using Bioconductor packages and were used for further data analysis [7]. The analysis of differentially expressed genes between the Myc^{f/f} and Myc^{Δ/Δ}/Ncr1^{Cre} group was performed using the negative binomial statistical model of read counts as implemented in the edgeR Bioconductor package^{[8]}. The pathway enrichment analysis was performed using The Database for Annotation, Visualization and Integrated Discovery (DAVID) gene functional classification tool^{[9]}. Enrichment analysis of translation-associated gene sets (REACTOME_TRANSLATION) from MSigDB (Broad Institute, Inc., Massachusetts Institute of Technology, and Regents of the University of California) was performed using gene set enrichment analysis (GSEA)^{[10]}. The number of permutations was 1,000. The signal-to-noise method was used. The raw RNA-seq sequencing data reported in this paper have been deposited in the Gene Expression Omnibus database under GSE142685.

Immunohistochemistry Staining. The formalin-fixed, paraffin-embedded tumor tissue sections were used for immunohistochemistry staining or hematoxylin and eosin (H&E) staining. For the former, samples were stained with anti-CD8 antibody (EPR20305, ab209775, Abcam, Cambridge, MA) or anti-NK1.1 antibody (ab197979, Abcam, Cambridge, MA). A Biotin Link was used as secondary antibody followed by streptavidin-peroxidase method, visualized with the DAB chromogen, and finally counter-stained with hematoxylin. The percentages of positive-staining cells were counted with at least four representative fields at 400× magnification by two individual researchers independently. Scoring of tumor stroma area is based on methods reported beforef. Human melanomal-1-PE tissues were purchased from BioCore USA, and were stained with anti-RSPO3 (17193-1-AP, ProteinTech, Rosemont, USA) and anti-CD31 (ab28364, Abcam, Cambridge, MA).

Isolation of Lymphocytes. Human peripheral blood samples of healthy donors were obtained from the Cell Processing Core and studies were approved by Institutional Review Board at Cincinnati Children's Hospital Medical Center. Peripheral blood mononuclear cells and granulocytes were obtained by Ficoll (07801, STEMCELL, Cambridge, MA) processing based on the manufacture's instruction. Lymphocytes were purified by magnetically labeling with human NK Cell Isolation Kit (130-092-657), human CD19 MicroBeads (130-050-301), human CD4+ T Cell Isolation Kit (130-096-533), and human CD8⁺ T Cell Isolation Kit (130-096-495) purchased from Miltenyi Biotech and sorted with an autoMACS Pro Separator.

Western Blotting. Human peripheral blood immune cells were purified with magnetic selection. The cell pellets were then lysed in sodium dodecyl sulfate (SDS) sample buffer containing 10 mM NaF, 10 mM β-Glycerophosphate, 1 mM phenylmethylsulfonyl fluoride, 0.2 mM Na3VO4, 2.5 mM dithiothreitol, 5% 2-mercaptoethanol, 1 mM 4-Amidinophenylmethanesulfonyl Fluoride Hydrochloride, and proteinase inhibitors followed sonication. Samples were boiled at 95° C. for 5 minutes and loaded to SDS-polyacrylamide gel electrophoresis (PAGE). The separated proteins were transferred to polyvinylidene fluoride (PVDF) membranes (Millipore, Merck KGaA, Darmstadt, Germany) and blocked with 5% BSA in PBST for 1 h at room temperature. The membranes were further probed with the indicated primary antibodies overnight at 4° C. The following primary antibodies were used: anti-LGR6 antibody (ab126747, Abcam, Cambridge, MA), anti-RSPO3 antibody (17193-1-AP, ProteinTech, Rosemont, USA), and anti-β-actin antibody (ab49900, Abcam, Cambridge, MA). Horseradish peroxidase-conjugated antibody to rabbit (NA934V, GE Healthcare) was used to detect primary antibodies using the Super Signal West Dura Chemiluminescent Substrate (Pierce) was used for ECL detection. Band intensity quantification was determined using Image Lab (version 5.2.1) software. All images presented are representative of two to three independent experiments.

NK Cell Cytotoxicity Assay. Tumor tissues were digested into single-cell suspension as shown in the section of Flow Cytometry and Cell Sorting and further processed with Ficoll (07801, STEMCELL, Cambridge, MA) to remove dead cells. Tumor-infiltrating NK cells were isolated using CD49b (DX5) MicroBeads, mouse (Miltenyi Biotec, 130-052-501) according to the manufacturer's instructions by an autoMACS Pro Separator (Miltenyi Biotec). B16F10 or YAC1 target cells were labeled for 2 hour with 2 μCi of ⁵¹Cr per 1×10⁴ target cells at 37° C., 5% CO2. Washing procedures were performed to remove excess ⁵¹Cr. Labeled target cells were added to 96-well round-bottom plates (1×10⁴ cells/well). Isolated NK cells were added to the plates with E:T ratios ranged between 50:1 and 6:1. The amount of 51Cr released, which corresponds to target cell death, was measured by a gamma scintillation counter. The percent cytotoxicity against target cells was calculated as: ((experimental lysis−spontaneous lysis)/(maximal lysis−spontaneous lysis))×100. To determine maximal lysis, 51Cr-labeled target cells were treated with 3% Triton X for 4 hours. To determine spontaneous release, target cells without effector cells were used for the assay.

Cell Viability Assays. Cells were seeded in 96-well plates in triplicate at a density of 4000 cells/100 μL/well. Cell viability was assayed with Cell Counting Kit-8 reagent (Dojindo, Japan) based on manufacture's instruction, and the relative growth was calculated by normalizing to day 0 results.

Statistical Analysis. Statistical analyses were performed using Prism 8.0.1 software. Selections of all statistical analysis methods meet the assumptions of the tests. Equality of variances between the groups was statistically compared. Student's t-test or Welch's t-test were used for comparisons of two groups. ANOVA with multiple comparisons was used for three or more groups and tumor growth profiles. The log-rank test was used to determine statistical significance for overall survival data. Multivariant regression analysis was used to determine whether RSPO3 level is an independent factor affecting NK-cell signature in TCGA datasets. Unless specifically noted, all data are representative of >2 independent experiments. Data are shown as mean±s.d. unless otherwise noted. P<0.05 was considered statistically significant. P-value is shown if 0.05<P<0.1.

### REFERENCES

1. Souza-Fonseca-Guimaraes F, Cursons J, Huntington ND. The Emergence of Natural Killer Cells as a Major Target in Cancer Immunotherapy. Trends Immunol 2019; 40(2):142-158.
2. Barry K C, Hsu J, Broz M L, Cueto F J, Binnewies M, Combes A J, et al. A natural killer-dendritic cell axis defines checkpoint therapy-responsive tumor microenvironments. Nat Med 2018; 24(8):1178-1191.
3. Hsu J, Hodgins J J, Marathe M, Nicolai C J, Bourgeois-Daigneault M C, Trevino T N, et al. Contribution of N K cells to immunotherapy mediated by PD-1/P D-L1 blockade. J Clin Invest 2018; 128(10):4654-4668.
4. Dong W, Wu X, Ma S, Wang Y, Nalin A P, Zhu Z, et al. The Mechanism of Anti-P D-L1 Antibody Efficacy against P D-L1-Negative Tumors Identifies N K Cells Expressing P D-L1 as a Cytolytic Effector. Cancer Discov 2019; 9(10):1422-1437.
5. Cozar B, Greppi M, Carpentier S, Narni-Mancinelli E, Chiossone L, Vivier E. Tumor-Infiltrating Natural Killer Cells. Cancer Discov 2020.
6. Ramakrishnan A B, Cadigan K M. Wnt target genes and where to find them. F1000Res 2017; 6:746.
7. Galluzzi L, Spranger S, Fuchs E, Lopez-Soto A. WNT Signaling in Cancer Immunosurveillance. Trends Cell Biol 2019; 29(1):44-65.
8. Cichocki F, Valamehr B, Bjordahl R, Zhang B, Rezner B, Rogers P, et al. GSK3 Inhibition Drives Maturation of N K Cells and Enhances Their Antitumor Activity. Cancer Res 2017; 77(20):5664-5675.
9. Malladi S, Macalinao D G, Jin X, He L, Basnet H, Zou Y, et al. Metastatic Latency and Immune Evasion through Autocrine Inhibition of WNT. Cell 2016; 165(1):45-60.
10. Xiao Q, Wu J, Wang W J, Chen S, Zheng Y, Yu X, et al. DKK2 imparts tumor immunity evasion through beta-catenin-independent suppression of cytotoxic immune-cell activation. Nat Med 2018; 24(3):262-270.
11. Spranger S, Bao R, Gajewski T F. Melanoma-intrinsic beta-catenin signalling prevents anti-tumour immunity. Nature 2015; 523(7559):231-235.
12. Luke J J, Bao R, Sweis R F, Spranger S, Gajewski T F. WNT/beta-catenin Pathway Activation Correlates with Immune Exclusion across Human Cancers. Clin Cancer Res 2019; 25(10):3074-3083.
13. de Lau W, Barker N, Low T Y, Koo B K, Li V S, Teunissen H, et al. Lgr5 homologues associate with Wnt receptors and mediate R-spondin signalling. Nature 2011; 476(7360):293-297.
14. Carmon K S, Gong X, Lin Q, Thomas A, Liu Q. R-spondins function as ligands of the orphan receptors LGR4 and LGR5 to regulate Wnt/beta-catenin signaling. Proc Natl Acad Sci USA 2011; 108(28):11452-11457.
15. Nagano K. R-spondin signaling as a pivotal regulator of tissue development and homeostasis. Jpn Dent Sci Rev 2019; 55(1):80-87.
16. de Lau W, Peng W C, Gros P, Clevers H. The R-spondin/Lgr5/Rnf43 module: regulator of Wnt signal strength. Genes Dev 2014; 28(4):305-316.
17. Lebensohn A M, Rohatgi R. R-spondins can potentiate WNT signaling without LGRs. Elife 2018; 7.
18. Szenker-Ravi E, Altunoglu U, Leushacke M, Bosso-Lefevre C, Khatoo M, Thi Tran H, et al. RSPO2 inhibition of RNF43 and ZNRF3 governs limb development independently of LGR4/5/6. Nature 2018; 557(7706):564-569.
19. Snippert H J, Haegebarth A, Kasper M, Jaks V, van Es J H, Barker N, et al. Lgr6 marks stem cells in the hair follicle that generate all cell lineages of the skin. Science 2010; 327(5971):1385-1389.
20. Ren W, Lewandowski B C, Watson J, Aihara E, Iwatsuki K, Bachmanov A A, et al. Single Lgr5- or Lgr6-expressing taste stem/progenitor cells generate taste bud cells ex vivo. Proc Natl Acad Sci USA 2014; 111(46):16401-16406.
21. Fullgrabe A, Joost S, Are A, Jacob T, Sivan U, Haegebarth A, et al. Dynamics of Lgr6(+) Progenitor Cells in the Hair Follicle, Sebaceous Gland, and Interfollicular Epidermis. Stem Cell Reports 2015; 5(5):843-855.
22. Blaas L, Pucci F, Messal H A, Andersson A B, Josue Ruiz E, Gerling M, et al. Lgr6 labels a rare population of mammary gland progenitor cells that are able to originate luminal mammary tumours. Nat Cell Biol 2016; 18(12): 1346-1356.
23. Marcus A, Mao A J, Lensink-Vasan M, Wang L, Vance R E, Raulet D H. Tumor-Derived cGAMP Triggers a STING-Mediated Interferon Response in Non-tumor Cells to Activate the N K Cell Response. Immunity 2018; 49(4):754-763 e754.
24. Hegde S, Krisnawan V E, Herzog B H, Zuo C, Breden M A, Knolhoff B L, et al. Dendritic Cell Paucity Leads to Dysfunctional Immune Surveillance in Pancreatic Cancer. Cancer Cell 2020; 37(3):289-307 e289.
25. Tirosh I, Izar B, Prakadan S M, Wadsworth M H, 2nd, Treacy D, Trombetta J J, et al. Dissecting the multicellular ecosystem of metastatic melanoma by single-cell RNA-seq. Science 2016; 352(6282):189-196.
26. Elyada E, Bolisetty M, Laise P, Flynn W F, Courtois E T, Burkhart R A, et al. Cross-Species Single-Cell Analysis of Pancreatic Ductal Adenocarcinoma Reveals Antigen-Presenting Cancer-Associated Fibroblasts. Cancer Discov 2019; 9(8):1102-1123.
27. Schmiedel B J, Singh D, Madrigal A, Valdovino-Gonzalez A G, White B M, Zapardiel-Gonzalo J, et al. Impact of Genetic Polymorphisms on Human Immune Cell Gene Expression. Cell 2018; 175(6):1701-1715 e1716.
28. Mabbott N A, Baillie J K, Brown H, Freeman T C, Hume D A. An expression atlas of human primary cells: inference of gene function from coexpression networks. BMC Genomics 2013; 14:632.
29. Wu C, Jin X, Tsueng G, Afrasiabi C, Su A I. BioGPS: building your own mash-up of gene annotations and expression profiles. Nucleic Acids Res 2016; 44(D1):D313-316.
30. Collins P L, Cella M, Porter S I, Li S, Gurewitz G L, Hong H S, et al. Gene Regulatory Programs Conferring Phenotypic Identities to Human N K Cells. Cell 2019; 176(1-2):348-360 e312.
31. Heng T S, Painter M W, Immunological Genome Project C. The Immunological Genome Project: networks of gene expression in immune cells. Nat Immunol 2008; 9(10):1091-1094.
32. Choi J, Baldwin T M, Wong M, Bolden J E, Fairfax K A, Lucas E C, et al. Haemopedia RNA-seq: a database of gene expression during haematopoiesis in mice and humans. Nucleic Acids Res 2019; 47(D1):D780-D785.
33. de Lau W B, Snel B, Clevers H C. The R-spondin protein family. Genome Biol 2012; 13(3):242.
34. Spits H, Artis D, Colonna M, Diefenbach A, Di Santo J P, Eberl G, et al. Innate lymphoid cells—a proposal for uniform nomenclature. Nat Rev Immunol 2013; 13(2):145-149.
35. Niida A, Hiroko T, Kasai M, Furukawa Y, Nakamura Y, Suzuki Y, et al. DKK1, a negative regulator of Wnt signaling, is a target of the beta-catenin/TCF pathway. Oncogene 2004; 23(52):8520-8526.
36. Whitfield J R, Beaulieu M E, Soucek L. Strategies to Inhibit Myc and Their Clinical Applicability. Front Cell Dev Biol 2017; 5:10.
37. Kim K A, Wagle M, Tran K, Zhan X, Dixon M A, Liu S, et al. R-Spondin family members regulate the Wnt pathway by a common mechanism. Mol Biol Cell 2008; 19(6):2588-2596.
38. Leushacke M, Barker N. Lgr5 and Lgr6 as markers to study adult stem cell roles in self-renewal and cancer. Oncogene 2012; 31(25):3009-3022.
39. Hao H X, Xie Y, Zhang Y, Charlat O, Oster E, Avello M, et al. ZNRF3 promotes Wnt receptor turnover in an R-spondin-sensitive manner Nature 2012; 485(7397):195-200.
40. Nusse R, Clevers H. Wnt/beta-Catenin Signaling, Disease, and Emerging Therapeutic Modalities. Cell 2017; 169(6):985-999.
41. Loftus R M, Assmann N, Kedia-Mehta N, O'Brien K L, Garcia A, Gillespie C, et al Amino acid-dependent cMyc expression is essential for N K cell metabolic and functional responses in mice. Nat Commun 2018; 9(1):2341.
42. Dong H, Adams N M, Xu Y, Cao J, Allan D S J, Carlyle J R, et al. The IRE1 endoplasmic reticulum stress sensor activates natural killer cell immunity in part by regulating c-Myc. Nat Immunol 2019; 20(7):865-878.
43. Zakiryanova G K, Kustova E, Urazalieva N T, Baimuchametov E T, Nakisbekov N N, Shurin M R. Abnormal Expression of c-Myc Oncogene in N K Cells in Patients with Cancer. Int J Mol Sci 2019; 20(3).
44. Fehniger T A, Cai S F, Cao X, Bredemeyer A J, Presti R M, French A R, et al. Acquisition of murine N K cell cytotoxicity requires the translation of a pre-existing pool of granzyme B and perforin mRNAs. Immunity 2007; 26(6):798-811.
45. Wagner J A, Wong P, Schappe T, Berrien-Elliott M M, Cubitt C, Jaeger N, et al. Stage-Specific Requirement for Eomes in Mature N K Cell Homeostasis and Cytotoxicity. Cell Rep 2020; 31(9):107720.
46. Sigal M, Logan C Y, Kapalczynska M, Mollenkopf H J, Berger H, Wiedenmann B, et al. Stromal R-spondin orchestrates gastric epithelial stem cells and gland homeostasis. Nature 2017; 548(7668):451-455.
47. Seshagiri S, Stawiski E W, Durinck S, Modrusan Z, Storm E E, Conboy C B, et al. Recurrent R-spondin fusions in colon cancer. Nature 2012; 488(7413):660-664.
48. Storm E E, Durinck S, de Sousa e Melo F, Tremayne J, Kljavin N, Tan C, et al. Targeting PTPRK-RSPO3 colon tumours promotes differentiation and loss of stem-cell function. Nature 2016; 529(7584):97-100.
49. Han T, Schatoff E M, Murphy C, Zafra M P, Wilkinson J E, Elemento O, et al. R-Spondin chromosome rearrangements drive Wnt-dependent tumour initiation and maintenance in the intestine. Nat Commun 2017; 8:15945.
50. Hilkens J, Timmer N C, Boer M, Ikink G J, Schewe M, Sacchetti A, et al. RSPO3 expands intestinal stem cell and niche compartments and drives tumorigenesis. Gut 2017; 66(6):1095-1105.
51. Zenkova D. K V, Sablina R., Artyomov M., Sergushichev A. Phantasus: visual and interactive gene expression analysis. https://genomeilfmoru/phantasus:doi: 10.18129/B18129.bioc.phantasus.
52. Tang Z, Kang B, Li C, Chen T, Zhang Z. GEPIA2: an enhanced web server for large-scale expression profiling and interactive analysis. Nucleic Acids Res 2019; 47(W1):W556-W560.
53. Jerby-Arnon L, Shah P, Cuoco M S, Rodman C, Su M J, Melms J C, et al. A Cancer Cell Program Promotes T Cell Exclusion and Resistance to Checkpoint Blockade. Cell 2018; 175(4):984-997 e924.
54. Afgan E, Baker D, Batut B, van den Beek M, Bouvier D, Cech M, et al. The Galaxy platform for accessible, reproducible and collaborative biomedical analyses: 2018 update. Nucleic Acids Res 2018; 46(W1):W537-W544.
55. Zenkova D., Kamenev V., Sablina R., Artyomov M., Sergushichev A. Phantasus: visual and interactive gene expression analysis. https://genome.ifmo.ru/phantasus doi: 10.18129/B9.bioc.phantasus.
56. Trapnell C, Pachter L, Salzberg S L. TopHat: discovering splice junctions with RNA-Seq. Bioinformatics 2009; 25(9):1105-1111.
57. Amezquita R A, Lun A T L, Becht E, Carey V J, Carpp L N, Geistlinger L, et al. Orchestrating single-cell analysis with Bioconductor. Nat Methods 2020; 17(2):137-145.
58. Anders S, McCarthy D J, Chen Y, Okoniewski M, Smyth G K, Huber W, et al. Count-based differential expression analysis of RNA sequencing data using R and Bioconductor. Nat Protoc 2013; 8(9):1765-1786.
59. Huang D W, Sherman B T, Tan Q, Collins J R, Alvord W G, Roayaei J, et al. The DAVID Gene Functional Classification Tool: a novel biological module-centric algorithm to functionally analyze large gene lists. Genome Biol 2007; 8(9):R183.
60. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA 2005; 102(43):15545-15550.
61. van Pelt G W, Kjaer-Frifeldt S, van Krieken J, Al Dieri R, Morreau H, Tollenaar R, et al. Scoring the tumor-stroma ratio in colon cancer:procedure and recommendations. Virchows Arch 2018; 473(4):405-412.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method for treating a cancer in an individual, comprising administering an effective amount of an R-spondin protein to said individual.

2. The method of claim 1, wherein said R-spondin protein is selected from R-spondin1, R-spondin2, R-spondin3, R-spondin4, and combinations thereof.

3. The method of claim 1, wherein said R-spondin protein comprises at least 95% sequence identity, or at least 90% sequence identity, or at least 85% sequence identity, or at least 80% sequence identity to at least one of R-spondin1, R-spondin2, R-spondin3, and R-spondin4.

4. The method of claim 1, wherein said R-spondin protein is a fragment of a full-length R-spondin protein.

5. The method of claim 4 wherein said fragment comprises a furin-1 domain.

6. The method of claim 4 wherein said fragment comprises a furin-1 domain and a furin-2 domain.

7. The method of claim 4 wherein said fragment comprises a furin-1 domain, and thrombospondin type 1 (TSP) and basic region (BR) (TSP/BR) domains.

8. The method of claim 4 wherein said fragment comprises a furin-1 domain, a furin-2 domain, and a thrombospondin type 1 (TSP) domain and a basic region (BR) (TSP/BR) domain.

9. The method of claim 4 wherein said fragment binds to Leucine-rich repeat-containing G-protein coupled receptor 4 (LGR4), Leucine-rich repeat-containing G-protein coupled receptor 5 (LGR5), and/or Leucine-rich repeat-containing G-protein coupled receptor 6 (LGR6).

10. The method of claim 1, wherein said R-spondin protein is administered via an R-spondin precursor.

11. The method of claim 10 wherein said R-spondin precursor comprises DNA, RNA or a combination thereof, wherein said R-spondin precursor expresses R-spondin in vivo after administration to said individual.

12. The method of claim 1, wherein said R-spondin precursor comprises an expression vector operably linked to an R-spondin gene or gene fragment, wherein said expression vector expresses R-spondin or gene fragment in vivo.

13. The method of claim 1, wherein said R-spondin protein or said R-spondin precursor is administered in an amount of from about 5 μg/kg to about 500 μg/kg, or from about 10 μg/kg to about 250 μg/kg, or from about 25 μg/kg to about 100 μg/kg, about 50 μg/kg tumor weight.

14. The method of claim 1, wherein said R-spondin protein or said R-spondin precursor is administered in a sterile saline solution.

15. The method of claim 1, wherein said R-spondin protein or said R-spondin precursor is administered at an interval selected from daily, every two days, every three days, every four days, every five days, every six days, every seven days, every two weeks, every three weeks, and monthly.

16. The method of claim 1 wherein said administering improves a prognosis in said individual diagnosed with said cancer, said cancer being selected from Skin Cutaneous Melanoma (SKCM), pancreatic adenocarcinoma (PAAD), lung squamous cell carcinoma (LUSC), and head and neck squamous carcinoma (HNSC), breast invasive carcinoma (BRCA), and cholangiocarcinoma (CHOL) Breast invasive carcinoma (BRCA), Thyroid carcinoma (THCA), Bladder Urothelial Carcinoma (BLCA), Colon adenocarcinoma (COAD), or Uveal Melanoma (UVM).

17. The method of claim 1, said administering being to a site of the cancer.

18. The method of claim 1, wherein said cancer forms a solid tumor, and wherein said administering is via intravenous injection or intra-tumor injection.

19. The method of claim 1, comprising administering an immune checkpoint inhibitor.

20. The method claim 1, comprising administering a checkpoint inhibitor, wherein said checkpoint inhibitor is administered at a time selected from prior to said R-spondin protein administration, during said R-spondin protein administration, after said R-spondin protein administration, or a combination thereof.

21-52. (canceled)
